SEQUENCE LISTING (81 sequences)

SEQ ID NO: 1 - protein, 8 aa, Homo sapiens
SIINFEKL

SEQ ID NO: 2 - protein, 9 aa, Homo sapiens
TMTRVLQGV

SEQ ID NO: 3 - protein, 9 aa, Homo sapiens
VLQGVLPAL

SEQ ID NO: 4 - protein, 9 aa, Homo sapiens
GVLPALPQV

SEQ ID NO: 5 - protein, 9 aa, Homo sapiens
KIFGSLAFL

SEQ ID NO: 6 - protein, 9 aa, Homo sapiens
EVDPIGHLY

SEQ ID NO: 7 - protein, 10 aa, Homo sapiens
GPRTAALGLL

SEQ ID NO: 8 - protein, 9 aa, Homo sapiens
LLGRNSFEV

SEQ ID NO: 9 - protein, 9 aa, Homo sapiens
VLMTEDIKL

SEQ ID NO: 10 - protein, 9 aa, Homo sapiens
YLLPAIVHI

SEQ ID NO: 11 - protein, 9 aa, Homo sapiens
TLAYLIFCL

SEQ ID NO: 12 - protein, 8 aa, Homo sapiens
YLEPGPVT

SEQ ID NO: 13 - protein, 9 aa, Homo sapiens
SLLMWITQV

SEQ ID NO: 14 - protein, 9 aa, Homo sapiens
ILAKFLHWL

SEQ ID NO: 15 - protein, 9 aa, Homo sapiens
AAGIGILTV

SEQ ID NO: 16 - protein, 9 aa, Homo sapiens
AIMDKNIIL

SEQ ID NO: 17 - protein, 9 aa, Homo sapiens
ALGIGILTV

SEQ ID NO: 18 - protein, 9 aa, Homo sapiens
ALMPVLNQV

SEQ ID NO: 19 - protein, 9 aa, Homo sapiens
ATDFKFAMY

SEQ ID NO: 20 - protein, 9 aa, Homo sapiens
ATTNILEHY

SEQ ID NO: 21 - protein, 9 aa, Homo sapiens
AVLPPLPQV

SEQ ID NO: 22 - protein, 9 aa, Homo sapiens
EADPTGHSY

SEQ ID NO: 23 - protein, 10 aa, Homo sapiens
ELTLGEFLKL

SEQ ID NO: 24 - protein, 11 aa, Homo sapiens
FLAEDALIITV

SEQ ID NO: 25 - protein, 10 aa, Homo sapiens
FLSTLTIDGV

SEQ ID NO: 26 - protein, 9 aa, Homo sapiens
FLSELTQQL

SEQ ID NO: 27 - protein, 9 aa, Homo sapiens
FLYDDNQRV

SEQ ID NO: 28 - protein, 9 aa, Homo sapiens
GILGFVFTL

SEQ ID NO: 29 - protein, 9 aa, Homo sapiens
GLNEEIARV

SEQ ID NO: 30 - protein, 9 aa, Homo sapiens
GVLPNIQAV

SEQ ID NO: 31 - protein, 10 aa, Homo sapiens
GVYDGEEHSV

SEQ ID NO: 32 - protein, 9 aa, Homo sapiens
IADMGHLKY

SEQ ID NO: 33 - protein, 9 aa, Homo sapiens
ILDQKINEV

SEQ ID NO: 34 - protein, 9 aa, Homo sapiens
ILKEPVHGV

SEQ ID NO: 35 - protein, 9 aa, Homo sapiens
ILNSRPPSV

SEQ ID NO: 36 - protein, 9 aa, Homo sapiens
IMDQVPFSV

SEQ ID NO: 37 - protein, 9 aa, Homo sapiens
IPSIQSRGL

SEQ ID NO: 38 - protein, 9 aa, Homo sapiens
ITDQVPFSV

SEQ ID NO: 39 - protein, 9 aa, Homo sapiens
ITNSRPPSV

SEQ ID NO: 40 - protein, 9 aa, Homo sapiens
KIFGALAFL

SEQ ID NO: 41 - protein, 9 aa, Homo sapiens
KIFGGLAFL

SEQ ID NO: 42 - protein, 9 aa, Homo sapiens
KIFGKLAFL

SEQ ID NO: 43 - protein, 9 aa, Homo sapiens
KIGEGTYGV

SEQ ID NO: 44 - protein, 16 aa, Homo sapiens
KKLLTQHFVQENYLEY

SEQ ID NO: 45 - protein, 9 aa, Homo sapiens
KLGEGTYGV

SEQ ID NO: 46 - protein, 9 aa, Homo sapiens
KLMSPKLYV

SEQ ID NO: 47 - protein, 9 aa, Homo sapiens
KLQELNYNL

SEQ ID NO: 48 - protein, 9 aa, Homo sapiens
KVLEYVIKV

SEQ ID NO: 49 - protein, 9 aa, Homo sapiens
LKMESLNFI

SEQ ID NO: 50 - protein, 9 aa, Homo sapiens
LPFDRTTVM

SEQ ID NO: 51 - protein, 9 aa, Homo sapiens
NAITNAKII

SEQ ID NO: 52 - protein, 9 aa, Homo sapiens
NLVPMVATV

SEQ ID NO: 53 - protein, 9 aa, Homo sapiens
QPEWFRNIL

SEQ ID NO: 54 - protein, 9 aa, Homo sapiens
QPEWFRNVL

SEQ ID NO: 55 - protein, 9 aa, Homo sapiens
RMFPNAPYL

SEQ ID NO: 56 - protein, 9 aa, Homo sapiens
RPYSNVSNL

SEQ ID NO: 57 - protein, 9 aa, Homo sapiens
SIGGVFTSV

SEQ ID NO: 58 - protein, 9 aa, Homo sapiens
SLFLGILSV

SEQ ID NO: 59 - protein, 9 aa, Homo sapiens
SLLMWITQC

SEQ ID NO: 60 - protein, 9 aa, Homo sapiens
SLLEKREKT

SEQ ID NO: 61 - protein, 9 aa, Homo sapiens
STAPPAHGV

SEQ ID NO: 62 - protein, 9 aa, Homo sapiens
STPPPGTRV

SEQ ID NO: 63 - protein, 9 aa, Homo sapiens
SVGGVFTSV

SEQ ID NO: 64 - protein, 9 aa, Homo sapiens
SYIGSINNI

SEQ ID NO: 65 - protein, 9 aa, Homo sapiens
TLHEYMLDL

SEQ ID NO: 66 - protein, 9 aa, Homo sapiens
TLQDIVLHL

SEQ ID NO: 67 - protein, 9 aa, Homo sapiens
TMMRVLQAV

SEQ ID NO: 68 - protein, 9 aa, Homo sapiens
TPQSNRPVM

SEQ ID NO: 69 - protein, 9 aa, Homo sapiens
VLQAVLPPL

SEQ ID NO: 70 - protein, 9 aa, Homo sapiens
VLQELNVTV

SEQ ID NO: 71 - protein, 10 aa, Homo sapiens
VMAGVGSPYV

SEQ ID NO: 72 - protein, 9 aa, Homo sapiens
YIFGSLAFL

SEQ ID NO: 73 - protein, 13 aa, Homo sapiens
YKYKVVKIEPLGV

SEQ ID NO: 74 - protein, 9 aa, Homo sapiens
YLEPGPVTA

SEQ ID NO: 75 - protein, 9 aa, Homo sapiens
YLEPGPVTV

SEQ ID NO: 76 - protein, 9 aa, Homo sapiens
YLLEMLWRL

SEQ ID NO: 77 - protein, 10 aa, Homo sapiens
YMLDLQPETT

SEQ ID NO: 78 - protein, 10 aa, Homo sapiens
RLDDDGNFQL

SEQ ID NO: 79 - protein, 9 aa, Homo sapiens
ATWAENIQV

SEQ ID NO: 80 - protein, 9 aa, Homo sapiens
YTMDGEYRL

SEQ ID NO: 81 - protein, 9 aa, Homo sapiens
SLTSINVQA

(19) United States
(12) Patent Application Publication
(10) Pub. No.: US 2014/0141455 A1
(43) Pub. Date: May 22, 2014

(54) METHODS OF ASSAYING VACCINE POTENCY

(71) Applicant: Receptor Logic, Inc., Austin, TX (US)

(72) Inventor: Jon A. Weidanz, Abilene, TX (US)

(21) Appl. No.: 14/053,012

(22) Filed: Oct. 14, 2013

Related U.S. Application Data
(63) Continuation of application No. 12/380,136, filed on Feb. 24, 2009, now abandoned.
Application No. 12/380,136 is a continuation-in-part of application No. 12/196,885, filed on Aug. 22, 2008, now abandoned.
Application No. 12/196,885 is a continuation-in-part of application No. 11/809,895, filed on Jun. 1, 2007, now abandoned.
Application No. 11/809,895 is a continuation-in-part of application No. 11/517,516, filed on Sep. 7, 2006, now abandoned.
Application No. 11/517,516 is a continuation-in-part of application No. 11/140,644, filed on May 27, 2005.
(60) Provisional application No. 61/061,534, filed on Jun. 13, 2008.
(60) Provisional application No. 61/191,871, filed on Sep. 12, 2008.
(60) Provisional application No. 60/965,766, filed on Aug. 22, 2007.
(60) Provisional application No. 60/965,766, filed on Aug. 22, 2007.
(60) Provisional application No. 60/810,079, filed on Jun. 1, 2006.
(60) Provisional application No. 60/714,621, filed on Sep. 7, 2005.
(60) Provisional application No. 60/751,542, filed on Dec. 19, 2005.
(60) Provisional application No. 60/752,737, filed on Dec. 20, 2005.
(60) Provisional application No. 60/838,276, filed on Aug. 17, 2006.
(60) Provisional application No. 60/574,857, filed on May 27, 2004.
(60) Provisional application No. 60/640,020, filed on Dec. 28, 2004.
(60) Provisional application No. 60/646,338, filed on Jan. 24, 2005.
(60) Provisional application No. 60/673,296, filed on Apr. 20, 2005.

(51) Int. Cl. G01N 33/569

(52) U.S. Cl. CPC G01N 33/56977
USPC 435/7.24

(57) ABSTRACT

Methods of assaying potency of a vaccine composition are provided. Said methods utilize a T cell receptor mimic that is reactive against a specific peptide/MHC complex. The potency of the vaccine is determined based upon the measured density of specific peptide/MHC complex present on the surface of the vaccine-treated antigen presenting cell.

### CROSS REFERENCE TO RELATED APPLICATIONS/INCORPORATION BY REFERENCE STATEMENT

This application is a continuation of U.S. Ser. No. 12/380,136, filed Feb. 24, 2009; which claims benefit under 35 U.S.C. 119(e) of US provisional application U.S. Ser. No. 61/061,534, filed Jun. 13, 2008; and U.S. Ser. No. 61/191,871, filed Sep. 12, 2008. The '136 application is also a continuation-in-part of U.S. Ser. No. 12/196,885, filed Aug. 22, 2008, now abandoned; which claims benefit under 35 U.S.C. 119(e) of US provisional application U.S. Ser. No. 60/965,766, filed Aug. 22, 2007. The '885 application is also a continuation-in-part of U.S. Ser. No. 11/809,895, filed Jun. 1, 2007, now abandoned; which claims benefit under 35 U.S.C. 119(e) of U.S. Ser. No. 60/810,079, filed Jun. 1, 2006. The '895 application is also a continuation-in-part of U.S. Ser. No. 11/517,516, filed Sep. 7, 2006, now abandoned; which claims benefit under 35 U.S.C. 119(e) of provisional applications U.S. Ser. No. 60/714,621, filed Sep. 7, 2005; U.S. Ser. No. 60/751,542, filed Dec. 19, 2005; U.S. Ser. No. 60/752,737, filed Dec. 20, 2005; and U.S. Ser. No. 60/838,276, filed Aug. 17, 2006. The '516 application is also a continuation-in-part of U.S. Ser. No. 11/140,644, filed May 27, 2005, now abandoned; which claims benefit under 35 U.S.C. 119(e) of provisional applications U.S. Ser. No. 60/574,857, filed May 27, 2004; U.S. Ser. No. 60/640,020, filed Dec. 28, 2004; U.S. Ser. No. 60/646,338, filed Jan. 24, 2005; and U.S. Ser. No. 60/673,296, filed Apr. 20, 2005. The entire contents of each of the above-referenced patents and patent applications are hereby expressly incorporated herein by reference.

### STATEMENT REGARDING FEDERALLY SPONSORED RESEARCH OR DEVELOPMENT

This inventive concept(s) was made with government support under Grant Number 70NANB4H3048 awarded by the Advanced Technology Program of the National Institute of Standards and Technology. The government has certain rights in the inventive concept(s).

### BACKGROUND

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal milieu of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often have deleterious and even lethal effects on the host (e.g., human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens. The extermination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events during cell differentiation and maturation resulting from allelic diversity of the parents; as such, hundreds of different HLA types exist throughout the world's population, resulting in a large immunological diversity. Such extensive HLA diversity throughout the population is the root cause of tissue or organ transplant rejection between individuals as well as of differing individual susceptibility and/or resistance to infectious diseases. HLA molecules also contribute significantly to autoimmunity and cancer.

Class I MHC molecules alert the immune response to disorders within host cells. Peptides which are derived from viral- and tumor-specific proteins within the cell are loaded into the class I molecule's antigen binding groove in the endoplasmic reticulum of the cell and subsequently carried to the cell surface. Once the class I MHC molecule and its loaded peptide ligand are on the cell surface, the class I molecule and its peptide ligand are accessible to cytotoxic T lymphocytes (CTL). CTLs survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic scientific perspective, many outstanding questions remain in the art regarding peptide presentation. For instance, it has been demonstrated that a virus can preferentially block expression of HLA class I molecules from a given locus while leaving expression at other loci intact. Similarly, there are numerous reports of cancerous cells that downregulate the expression of class I HLA at particular loci. However, there is no data describing how (or if) the classical HLA class I loci differ in the peptides they bind. It is therefore unclear how class I molecules from the different loci vary in their interaction with viral- and tumor-derived ligands and the number of peptides each will present.

Discerning virus- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides (Cox et al., 1994). This approach has been employed to identify peptide ligands specific to cancerous cells. A second technique utilizes predictive algorithms to identify peptides capable of binding to a particular class I molecule based upon previously determined motif and/or individual ligand sequences (De Groot et al., 2001); however, there have been reports describing discrepancies between these algorithms and empirical data. Peptides having high predicted probability of binding from a pathogen of interest can then be synthesized and tested for T cell reactivity in various assays, such as but not limited to, precursor, tetramer and ELISpot assays.

Many cancer cells display tumor-specific peptide-HLA complexes derived from processing of inappropriately expressed or overexpressed proteins, called tumor associated antigens (TAAs) (Bernhard et al., 1996; Baxevanis et al., 2006; and Andersen et al., 2003). With the discovery of mAb technology, it was believed that “magic bullets” could be developed which specifically target malignant cells for destruction. Current strategies for the development of tumor specific antibodies rely on creating monoclonal antibodies (mAbs) to TAAs displayed as intact proteins on the surface of malignant cells. Though targeting surface tumor antigens has resulted in the development of several successful anti-tumor antibodies (Herceptin and Rituxan), a significant number of patients (up to 70%) are refractory to treatment with these antibody molecules. This has raised several questions regarding the rationale for targeting whole molecules displayed on the tumor cell surface for developing cancer therapeutic reagents. First, antibody-based therapies directed at surface antigens are often associated with lower than expected killing efficiency of tumor cells. Free tumor antigens shed from the surface of the tumor occupy the binding sites of the anti-tumor specific antibody, thereby reducing the number of active molecules and resulting in decreased tumor cell death. Second, current mAb molecules do not recognize many potential cancer antigens because these antigens are not expressed as an intact protein on the surface of tumor cells. The tumor suppressor protein p53 is a good example. p53 and similar intracellular tumor associated proteins are normally processed within the cell into peptides which are then presented in the context of either HLA class I or class II molecules on the surface of the tumor cell. Native antibodies are not generated against peptide-HLA complexes. Third, many of the antigens recognized by antibodies are heterogenic by nature, which limits the effectiveness of an antibody to a single tumor histology. For these reasons it is apparent that antibodies generated against surface expressed tumor antigens may not be optimal therapeutic targets for cancer immunotherapy.

Recent years have seen an increase in the development and testing of therapeutic cancer vaccines (Itoh et al., 2006; Markovic et al., 2006; and Hersey et al., 2005). Therapeutic vaccines for cancer and certain types of viral infections are aimed at stimulating cell-mediated immune responses, in particular those mediated by cytotoxic T lymphocytes (CTL) (Oka et al., 2006; Adotevi et al., 2006; and Xia et al., 2006). Therefore, the development of a cytotoxic effector arm of an anti-tumor response to vaccines requires that the epitopes be presented in the context of human leukocyte antigen (HLA) class I molecules on antigen-presenting cells. To date, several hundred human tumor-associated antigens (TAA) have been described (Novellino et al., 2005), but still the relationship between TAA expression, MHC-peptide density, recognition of tumor cells by CTL and eventual tumor cell lysis is not completely understood. Studies by the inventor have been unable to show any correlation between the expression of Her2/neu protein and the level of a dominant Her2/neu peptide presented by HLA-A2 on tumor cells (Weidanz et al., 2006). Furthermore, the experience with tumor antigens is that less than 50% of predicted peptides for which specific T cell receptor repertoire exists can actually be used to generate CTL that kill tumors in vitro (Clark et al., 2005). In the absence of efficient presentation of peptide-MHC on the surface of professional antigen-presenting cells, antigen-specific CTL priming can be minimal or virtually undetectable. Thus, the development of a potency assay that is rapid, consistent and easy to perform would be invaluable for assessing a vaccine's ability to elicit CTL responses.

One of the primary goals of a cancer vaccine is to elicit CTL responses, but the measurement of the potency of such responses has largely remained qualitative and semi-quantitative. Techniques such as flow cytometry and ELISA, although quantitative, only address the peptide binding properties and do not accurately reflect functional parameters involved in antigen uptake and processing by antigen-presenting cells such as Dendritic cells (DCs) and macrophages. The frequent discrepancy between antigen expression and specific epitope density suggests that a variety of scientific rationales need to be considered for experimental results to be meaningful (Weidanz et al., 2006). For instance, small animal challenge experiments in a prophylactic setting can be used but could be time-consuming and would require costly experiments to be conducted using large numbers of animals. Finally, CTL lines or clones and T cell hybridomas exposed to vaccine-treated cells are often used to assess epitope presentation by measuring cell proliferation, target cell lysis and cytokine production (Keilholz et al., 2006; and Whiteside et al., 2003). These assays, however, suffer from several limitations including but not limited to, inconsistent assay reproducibility and difficulty in producing and maintaining high quality reagents. In addition, the costs for maintaining eternal growth of cell-based reagents while providing quality assurance, overcoming assay bias and antigen specificity could be prohibitively high (Mosca et al., 2001; Petricciani et al., 2006; and Hinz et al., 2006). Therefore, there is a great need for the development of assays that can assess the potency of therapeutic products in the vaccine industry. The Food and Drug Administration (FDA) has defined potency as “the specific ability or capacity of a product to affect a given result” (Petricciani et al., 2006; and Keilholz et al., 2002). Therefore, the goal of potency assays is twofold: (1) to ensure that a given vaccine has at least a predefined minimum level of potential biological activity such as stimulation of antigen-specific CTL lines or clones and (2) that lot-to-lot consistency of the manufactured product can be readily monitored.

Recently it has been shown that the density of specific peptides displayed by MHC class I complexes directly correlates with the CTL response to virus and cancer (Bullock et al., 2000; and Wherry et al., 1999). In the study by Wherry et al., the authors used a recombinant vaccinia virus to deliver OVA peptide SIINFEKL (SEQ ID NO:1) to a murine fibroblast cell line and then quantitated the level of SIINFEKL peptide-MHC class I complexes using an anti-SIINFEKL peptide-K^{b} specific antibody (Wherry et al., 1999). Of note, the CTL-mediated cell lysis and cytokine release were directly dependent on the level of the specific epitope. The inventor has recently demonstrated a direct correlation between Her2/neu(369) peptide-HLA-A2 complexes and CTL cell lysis (Weidanz et al., 2006), and this result is consistent with the aforementioned studies. Collectively, these findings raise the possibility of measuring potency of CTL-inducing vaccines by using antibodies specific for peptide-MHC class I complexes.

Several investigators have produced antibodies for direct detection and visualization of specific peptide-MHC complexes on the surface of cells (Andersen et al., 1996; and Denkberg et al., 2002). Porgador et al. generated the 25.D1.16 mAb specific for the SIINFEKL (SEQ ID NO:1) peptide-K^{b} complex for visualizing such complexes in vivo in mice (Porgador et al., 1997). Using an analogous approach, Reiter's group isolated anti-peptide-MHC monoclonal antibodies from both mouse and human antibody phage display libraries (Denkberg et al., 2002; and Lev et al., 2002). In U.S. patent application Ser. No. 11/809,895, filed Jun. 1, 2007; U.S. Ser. No. 11/517,516, filed Sep. 7, 2006 (Publication No. US 2007/00992530 A1, published Apr. 26, 2007); and U.S. Ser. No. 11/140,644, filed May 27, 2005 (Publication No. US 2006/0034850 A1, published Feb. 16, 2006), the entire contents of which are hereby expressly incorporated herein by reference, the inventor has disclosed and claimed the generation of anti-MHC class I peptide monoclonal antibodies, called T cell receptor mimics (TCRm), as well as methods of producing same. These TCRm antibodies have high affinity and avidity for MHC-peptide complexes and are capable of detecting low densities of the specific MHC-peptide complex present on tumor cells.

Therefore, there is a need in the art for a method for assessing the potency of a vaccine composition that overcomes the disadvantages and defects of the prior art. It is to such method, and the compositions utilized in such method, that the presently disclosed and claimed inventive concept(s) is directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A graphically depicts that HLA class I molecules display peptides processed from intracellular proteins, and present said complex to T-cell receptors. Recognition of nonself peptides stimulates the cellular immune system to eliminate the diseased cell. FIG. 1B graphically depicts that T-Cell Receptor mimics (TCRm's) exhibit similar binding specificity to cytotoxic T-lymphocyte recognition of particular peptide-HLA complexes and act as a soluble reagent serving as an alternative to cell-based assays.

FIG. 2 illustrates a flow cytometry assay where T2 cells (lacking antigen presenting functions and presenting exogenously supplied peptides) are separately pulsed with either Peptide 1 (VLQGVLPAL; SEQ ID NO:3) or closely related Peptide 2 (VLQAVLPPL; SEQ ID NO:69) and then stained with a TCRm that was raised against the Peptide 1/HLA-A*0201 complex. A shift is only observed with cells pulsed with the cognate Peptide 1.

FIG. 3 graphically illustrates TCRm's show no cross reactivity to different HLA class I alleles. In this figure, a TCRm that is specific to a given peptide-HLA-A*0201 complex was examined. Said figure demonstrates that no binding occurs to the HLA allele itself without the presence of peptide-antigen, and also demonstrates that no non-specific binding occurs when exposed to different HLA class I alleles.

FIG. 4 graphically depicts affinity binding data for TCRm's RL08A and RL09A. Affinity determination for RL08A (left panel) and RL09A (right panel) was carried out on a SensiQ surface plasmon resonance instrument (ICX Nomadics, Oklahoma City, Okla., USA). In brief, protein A/G was coupled to a sensor chip to capture approximately 6 nM of either RL08A or RL09A antibody. FIG. 4A shows the binding affinity data for RL08A. Monomers of Gp100-peptide (SEQ ID NO:75)/HLA-A2 complexes were run over the sensor chip at concentrations of 12, 24, 48, 96, 192, 364 and 786 nM. Binding values were obtained with on- and off-rates of 2.275×10⁴ (M-1s-1) and 4.97×10⁻⁴ (s−1), respectively, resulting in a final KD of 21.8 nM. These values are approximately 3-fold lower than those reported by Denkberg et al. (Eur. J. Immunol, 2004; 34:2919), who found that their Gp100-peptide/HLA-A2 monoclonal antibody had a KD of 60 nM. Monomers of NY-ESO-1-peptide (SEQ ID NO:13)/HLA-A2 complexes were then passed over the RL09A coated chip at concentrations of 12, 24 and 48 nM. When measured by SensiQ, binding occurred with on- and off-rates of 2.158×10⁵ (M-1s-1) and 2.424×10⁻³ (s−1), respectively, resulting in a final KD (k_{off}/kₒₙ) of 11 nM as seen in FIG. 4B. Again, these values are approximately 3-fold lower than those reported by Denkberg et al. (PNAS, 2002; 99:9421), who found that their NY-ESO-1-peptide/HLA-A2 antibody had a KD of 30 nM.

FIG. 5 illustrates quantitative data from a flow cytometry assay, where T2 cells (which lack the ability to process antigens, but specifically load exogenous peptides) are pulsed with the appropriate peptide “A” (Gp100 peptide-YLEPGPVTV; SEQ ID NO:75), and the cognate TCRm (RL08A) is allowed to bind any presented complexes. The Mean Fluorescence Intensity (MFI) is measured using the shift in the sample flow cytometry peak compared with control TCRm antibodies and plotted in the table.

FIG. 6 illustrates a peptide titration study that demonstrates sensitivity of the T cell receptor mimic (TCRm) RL08A. An antigen presenting cell line was pulsed with decreasing amounts of relevant Gp100 peptide-YLEPGPVTV (SEQ ID NO:75) and then stained with a constant amount (250 ng/ml) of RL08A. Bound RL08A was detected using rat anti-mouse mAb-phycoerythrin (PE) conjugate and flow cytometric analysis. RL08A detection is dose-dependent and shown to be sensitive down to sub-nanomolar Gp100 peptide-YLEPGPVTV (SEQ ID NO:75) concentrations.

FIG. 7 graphically depicts PolyTest peptide competition assays for affinity determination of HLA-A*0201 peptide-epitopes. Two hCGβ peptides (TMT and GVL) were evaluated using a constant concentration of activated soluble HLA-A*0201 in the presence of 2.2 nM standard FITC-labeled peptide. After reaching equilibrium conditions, fluorescence polarization expressed in mP was measured. Values obtained at different peptide dilutions were graphed and inhibitory concentrations expressed as log [IC50]'s determined by fitting the data to a dose-response model. Results show that both epitopes are of high affinity with very similar binding strength.

FIG. 8 graphically depicts characterization of anti-hCGβ-HLA-A*0201 TCRm binding specificity. ELISA was performed in a plate coated with 0.1 μg of peptide-HLA-A*0201-tetramer complexes that included the following: TMT₍₄₀₎ (40-48, TMTRVLQGV; SEQ ID NO:2), VLQ₍₄₄₎ (44-52, VLQGVLPAL; SEQ ID NO:3), GVL₍₄₇₎ (47-55, GVLPALPQV; SEQ ID NO:4), and Her2/neu₍₃₆₉₎ (369-377, KIFGSLAFL; SEQ ID NO:5). Other control HLA class I complexes used in the binding assay included HLA-A*0101-tetramer complex loaded with EVDPIGHLY₍₁₆₁₎ (SEQ ID NO:6) from MAGE-3 cancer testis antigen and HLA-B*0702 monomer loaded with peptide GPRTAALGLL₍₄₎ (SEQ ID NO:7) from reticulocalbin protein. Binding specificity for TMT₍₄₀₎ and GVL₍₄₇₎ was determined by adding 0.25 μg of the following antibodies to wells: (A) 3F9 TCRm specific for TMT₍₄₀₎-HLA-A*0201 complex and (B) 1B10 TCRm specific for GVL₍₄₇₎-HLA-A*0201. Bound antibody was detected using a horseradish peroxidase (HRP)-conjugated goat anti-mouse IgG (1:5000 dilution), and color was developed with ABTS substrate. The absorbance was read at OD405 nm.

FIG. 9 graphically depicts characterization of anti-hCGβ TCRm mAbs for detection of TMT₍₄₀₎-HLA-A*0201 and GVL₍₄₇₎-HLA-A*0201 complexes on T2 cells. T2 cells were incubated with 20 μM of (A and B) TMT₍₄₀₎, VLQ₍₄₄₎ or GVL₍₄₇₎ peptides. Cells were then stained either with (A) 3F9 TCRm or IgG1 isotype control (filled area), or (B) 1B10 or isotype control (filled area). In all experiments bound antibody was detected using goat anti-mouse PE conjugate.

FIG. 10 graphically depicts that Vaccine-treated DCs elicit Ag-specific CTL response. Antigen-specific T cells were generated as described in Methods section. Briefly, DCs were either treated with vaccine or vehicle (control) and matured for 24 h with Poly I:C and then added to B11-hCGβ-specific CTL at a 1:1 ratio. Supernatant was collected at 24 and 48 h post-incubation and tested for interferon-γ production (10 pg/ml) using the BD OptEIA™ ELISA Kit II.

FIG. 11 graphically depicts inhibition of peptide-specific CTL lines using TCRm antibodies. hCGβ peptide-specific T cells were co-cultured with T2 cells as such or loaded with a specific hCGβ peptide (100 ng/ml) in the presence or absence of an HLA-A2.1-hCGβ peptide complex specific TCRm (50 ng/ml). Cytolytic granule granzyme-B production by Ag-specific CTL was measured in a GrB ELISpot assay. TMT peptide, SEQ ID NO:2; GVL peptide, SEQ ID NO:4.

FIG. 12 graphically depicts that DCs can cross-present HLA class I-restricted hCGβ epitopes to CD8+ T cells. Cytolytic T cells generated to hCGβ antigen by repeated stimulation with vaccine (20 μg/ml B11-hCGβ+poly IC (50 ng/ml)-activated DCs recognize cross-presented hCGβ epitopes. hCGβ-specific TCRm (50 ng/ml) only can effectively block a specific hCGβ-directed response since a TCRm to an unrelated antigen (NY-ESO-1) does not.

FIG. 13 graphically depicts that Vaccine-treated DCs reveal time-dependent presentation of CTL epitopes. Immature DCs were treated with vaccine (B11-hCGβ fusion protein) or with control vaccine (B11-CEA fusion protein) for up to 3 days before maturation with Poly I:C reagent (50 μg/ml). mDCs were then stained with TCRms, anti-TMTpeptide-HLA-A2 (3F9) and anti-GVL peptide-HLA-A2 (1B10). Detection of bound 3F9 and 1B10 was performed using a goat-anti-mouse-FITC conjugate.

FIG. 14 graphically depicts the characterization of TCRm binding detection sensitivity. T2 cells were incubated with decreasing concentrations (2000-0.150 nM) of (A) TMT peptide and (B) GVL peptide and stained with (A) 3F9 (B) 5E12, or (C) 4A3 TCRm-PE conjugates. The number of specific complexes was determined by plotting the TCRm staining intensity on to a standard curve generated using BD-CALIBRITE™ PE-beads. Numbers plotted above bars for peptide concentrations of 0.15 nM and 78 nM indicate the total specific peptide-HLA-A*0201 complexes detected on peptide-pulsed T2 cells.

FIG. 15 illustrates a time course analysis using vaccine containing Gp100 antigen: Gp100 peptide-YLEPGPVTV (SEQ ID NO:75) presentation. Antigen presenting cells were treated with vaccine containing Gp100 and subjected to intracellular staining with anti-Gp100 (purple shading—bottom 3 panels) as well as cell surface staining with RL08A (purple shading—top 3 panels) at 24 h, 48 h and 72 h post treatment. Separation from isotype control (green line) is shown by flow cytometry. TCRm-RL08A enables monitoring of de novo processing of Gp100, allowing for direct analysis of Gp100 processing kinetics and presentation of peptide-YLEPGPVTV/HLA-A2 complexes on the surface of vaccine treated antigen presenting cells. TCRm's offer this functionality with a variety of vaccine formats, including but not limited to: virus expression vectors, nucleic acid, microbial vectors, protein/peptide, and the like.

FIG. 16 illustrates peptide/HLA epitope presentation visualized by TCRm staining and immunocytochemistry. Antigen presenting cells were treated with vaccine containing Gp100 followed by incubation at 250 ng/ml with RL08A (left panel) and a control TCRm (right panel). Specific binding of RL08A to cells treated with vaccine containing Gp100 (left panel) was detected using a goat anti-mouse-FITC conjugate (green) and fluorescence microscopy. Dapi blue nuclear stain (right panel) was used to indicate the presence of antigen presenting cells attached to the glass slide.

FIG. 17 illustrates CTL activity and TCRm specificity for GP100 peptide-YLEPGPVTV (SEQ ID NO:75) and NY-ESO-1 peptide-SLLMWITQV (SEQ ID NO:13). Specificity of RL08A and RL09A was demonstrated in a competition assay where each respective TCRm was able to decrease CTL stimulation by blocking the T-cell receptor's ability to recognize and bind Gp100 peptide-YLEPGPVTV/HLA-A2 and NY-ESO-1 peptide-SLLMWITQV/HLA-A2 complexes. Blue bars represent cells without TCRm added and red bars represent addition of specific TCRm. Interferon-gamma cytokine production is significantly reduced at antigen dose levels of 1.0× and 0.1× (top & bottom right-side panels).

FIG. 18 illustrates that HLA-peptide complex density correlates with the level of CTL stimulation and intensity of TCRm binding. The level of direct binding of RL08A & RL09A to cognate peptide-antigen/HLA-A2 complexes on the surface of antigen presenting cells, represented as the change in Mean Fluorescence Intensity (ΔMFI), correlates with CTL stimulation assessed by the percentage of CD8+ T cells expressing interferon-gamma after incubation with vaccine treated antigen presenting cells.

FIG. 19 illustrates the benchmarking of TCRm staining of CTL stimulation. Using vaccine dosing studies, the minimal acceptable CTL stimulation activity was determined (blue bar) and set as acceptance threshold value (blue dashed line) for both Vaccine Antigens A (gp 100) and B (NYESO1). Parallel studies were carried out quantitating the number of specific HLA-peptide from gp100 and NYESO-1 complexes present on antigen presenting cells (purple and green bars, respectively). The complex numbers determined by TCRm staining of each antigen was determined at the threshold dose of each vaccine. The Established CTL threshold was used to derive Correlative TCRm staining thresholds. The complex numbers measured by TCRm RL8A binding gp100-derived peptides for Vaccine containing the gp100 Antigen at this Correlative threshold was ~450 HLA A*02-peptide a complexes (purple dashed line), and by RL9A for vaccine containing the NYESO-1 Antigen this value was ~700 HLA A*02-peptide b complexes (green dashed line). These Correlative threshold values of complexes, which have been benchmarked to CTL stimulation, now can be used to measure the potency of vaccine lots and formulations using appropriate archived standards.

FIG. 20 illustrates the use of CTL threshold as pass/fail criteria in the TCRm vaccine potency test of the presently disclosed and claimed inventive concept(s). The potency of nine different Gp 100 Vaccine formulations were compared using the TCRm quantitative potency assays measuring the numbers of HLA-Gp100 peptide complexes. A Gp100 vaccine standard was used to compare the various vaccine formulations and the CTL threshold for the Gp100 TCRm-RL08A, determined previously, was used as the pass/fail benchmark for the formulations. Using this basis, formulations 1 through 8 were deemed acceptable while formulation 9 failed based on the CTL activity threshold benchmark.

FIG. 21 graphically depicts three different batches of antigen presenting cells that were exposed to a constant dose of Gp100 antigen (Antigen “A”; SEQ ID NO:75) and assayed using RL08A-TCRm (TCRm #1) or control TCRm using flow cytometry. The ΔMFI values were calculated from the individual flow cytometry plots, averaged, and then presented graphically with standard deviation bars.

FIG. 22 illustrates TCRm staining adapted to QuantiBRITE™ PE bead system from BD Biosciences. Adaptation of TCRm staining readout from qualitative assay results to quantify specific peptide/HLA complexes/cell. Antigen presenting cells were treated with vaccine containing Gp100 and then stained with RL08A to determine the quantity of specific Gp100 peptide-YLEPGPVTV(SEQ ID NO:75)/HLA-A2 complexes present on the cell surface at 72 h post infection. Linear regression was performed using the geometric means of the four QuantiBRITE™ PE bead populations (low, medium low, medium high and high) and the mean number of PE molecules per bead (lot #05765) according to the manufacturer's instructions.

FIG. 23 illustrates quantitative measurement of peptide/HLA-A2 Gp100 epitope complexes. Antigen presenting cells treated with vaccine expressing Gp100 at doses of 10.0×, 1.0× and 0.1× and then stained with RL08A (blue) and RL09A (red/control) at 24 h, 48 h, 72 h and 96 h post-infection. Both TCRm's were used at [250 ng/mL]. Bound antibody was detected using rat anti-mouse IgG-PE conjugate. QuantiBRITE™ PE beads were run in parallel according to description given in FIG. 17. Linear regression was performed. Anti-isotype control antibody values are subtracted from the RL08A values. Results are plotted at molecules/cell (specific peptide/HLA complexes/cell) versus antigen dose.

FIG. 24 illustrates quantitative measurement of peptide/HLA-A2 NY-ESO-1 epitope complexes. Antigen presenting cells treated with vaccine expressing NY-ESO-1 at doses of 10.0×, 1.0× and 0.1× and then stained with RL09A (red) and RL08A (blue/control) at 24 h, 48 h, 72 h and 96 h post-infection. Both TCRm's were used at [250 ng/mL]. Bound antibody was detected using rat anti-mouse IgG-PE conjugate. QuantiBRITE™ PE beads were run in parallel according to description given in FIG. 10. Linear regression was performed. Anti-isotype control antibody values are subtracted from the RL09A values illustrating that detection of peptide-HLA complexes using TCRm's is possible down to the lowest tested multiplicity of infection (MOI) of 0.1 beginning as early as 24 h post-infection (top left panel). Results are plotted as molecules/cell (specific peptide/HLA complexes/cell) versus antigen dose.

FIG. 25 illustrates quantitative measurement of all HLA A*02 molecules. Antigen presenting cells were treated with two different doses of Gp100 antigen vaccine (Antigen “A”; SEQ ID NO:75) and harvested at 24, 48, 72 or 96 hours post treatment. The number of specific Gp100 antigen-peptide epitope complexes was quantified using the QuantiBRITE™ system and RL08A-TCRm. The total number of HLA A*02 molecules were quantified using an anti-HLA A*02 mAb and the QuantiBRITE™ system. The percentage of Gp100 antigen occupied HLA molecules were calculated and presented in graphical format.

FIG. 26 illustrates that TCRm's establish a quantitative baseline for ELISpot assays. ELISpot assay was conducted with the contents described below each individual sample result. The inclusion of the specific TCRm antibody reduces the assay background (in red) to virtually zero, whereas non-specific TCRm shows no effect. The significance between the sample with and without the vaccine is greatly enhanced by the inclusion of the TCRm antibody.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well-known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In one embodiment, oligonucleotides are 10 to 60 bases in length, such as but not limited to, 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides of the inventive concept(s) can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the inventive concept(s) selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the inventive concept(s) and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, such as at least 90 to 95 percent sequence identity, or at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disbustituted amino acids, N-alkyl amino acids, lactic acid, and other unconventional amino acids may also be suitable components for polypeptides of the presently disclosed and claimed inventive concept(s). Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, such as at least 90 percent sequence identity, or at least 95 percent sequence identity, or at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Particular conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the presently disclosed and claimed inventive concept(s), providing that the variations in the amino acid sequence maintain at least 75%, such as at least 80%, 90%, 95%, and 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More particular families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Particular amino- and carboxy-termini of fragments or analogs occur near boundaries of functional domains. Structural and functional domains can be identified by comparison of the nucleotide and/or amino acid sequence data to public or proprietary sequence databases. Particularly, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the inventive concept(s).

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (such as conservative amino acid substitutions) may be made in the naturally-occurring sequence (particularly in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure©. Branden and J. Tooze, eds., Garland Publishing, New York, N.Y. (1991)); and Thornton et al. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, such as at least 14 amino acids long or at least 20 amino acids long, usually at least 50 amino acids long or at least 70 amino acids long.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”.

The terms “MHC light chain” and “MHC heavy chain” as used herein will be understood to refer to portions of the MHC molecule. Structurally, class I molecules are heterodimers comprised of two noncovalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “β-2-microglobulin”, and “β2m” may be used interchangeably herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is <1 μM, or <100 nM, or <10 nM.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985).

An “isolated” antibody is one which has been identified and separated and/or recovered from a component of the environment in which it was produced. Contaminant components of its production environment are materials which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or silver stain. Isolated antibody includes the antibody in situ within recombinant cells since at least one component of the antibody's natural environment will not be present. Ordinarily, however, isolated antibody will be prepared by at least one purification step.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%.

The term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877). The more highly conserved portions of variable domains are called the framework (FR). The variable domains of native heavy and light chains each comprise four FR regions, largely adopting a 3-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)2 fragment that has two antigen binding fragments which are capable of cross-linking antigen, and a residual other fragment (which is termed pFc′). As used herein, “functional fragment” with respect to antibodies, refers to Fv, F(ab) and F(ab′)2 fragments.

An “Fv” fragment is the minimum antibody fragment which contains a complete antigen recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The heavy chains constant domains that correspond to the different classes of immunoglobulins are called α, Δ, ε, γ and μ, respectively. The subunit structures and three-dimensional configurations of different classes of immunoglobulins are well-known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed inventive concept(s) may be made by the hybridoma method first described by Kohler and Milstein, Nature 256, 495 (1975), or may be made by recombinant methods, e.g., as described in U.S. Pat. No. 4,816,567. The monoclonal antibodies for use with the presently disclosed and claimed inventive concept(s) may also be isolated from phage antibody libraries using the techniques described in Clackson et al. Nature 352: 624-628 (1991), as well as in Marks et al., J. Mol. Biol. 222: 581-597 (1991).

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The terms “label”, “detectable marker” and “detection moiety” are used interchangeably herein.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, such as more than about 85%, 90%, 95%, and 99%. In one embodiment, the object species is purified to essential homogeneity (contaminant species cannot be detected in the composition by conventional detection methods) wherein the composition consists essentially of a single macromolecular species.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

“Mammal” for purposes of treatment refers to any animal classified as a mammal, including human, domestic and farm animals, nonhuman primates, and zoo, sports, or pet animals, such as dogs, horses, cats, cows, etc.

The term “antigen presenting cell” as used herein will be understood to include any cell that can present peptides in the context of MHC molecules. In one embodiment, the antigen presenting cell must also be capable of processing proteins/polypeptides into peptides that may be presented in the context of MHC molecules. Examples of antigen presenting cells that may be utilized in accordance with the presently disclosed and claimed inventive concept(s) include, but are not limited to, dendritic cells (DCs), macrophages and B cells.

In the presently disclosed and claimed inventive concept(s), a system for assessing potency of a vaccine using an agent that quantitatively measures the number of specific peptide/MHC complexes on the surface of vaccine-treated cells is contemplated.

Active immunotherapy offers exciting prospects to direct the body's own immune responses to resolve localized or systemic disease. Antigen processing is central to active immunotherapy, whether the approach seeks to elicit cytotoxic T-lymphocyte (CTL) responses to treat cancer and intracellular pathogen infection, or if the goal is to induce T-cell anergy, removing T-cell subsets responsible for damaging autoimmune responses. Active immunotherapies most often require the intracellular expression of a disease-associated protein or antigen and processing through the Human Leukocyte Antigen (HLA) class I or class II system (also known as the Major Histocompatibility Complex; MHC). Antigen expression alone is insufficient to predict the activity of a given immunotherapy-appropriate antigen processing and presentation must be measured if the mode of action and associated potency of the immunotherapy can be addressed. Potency is important to measure in an immunotherapeutic product, especially at product release—to compare lot to lot variability and during stability analyses to insure time, transport and storage conditions have not compromised the drug product.

Present means of measuring active immunotherapy potency are generally qualitative, or semi-quantitative in nature. Antigen expression is most often used as initial potency measurements for product development and early stage clinical development. This method can be quantitative, however, it and other approaches including, labeling of cells using flow cytometry or traditional ELISA-based methods do not adequately address the mode of action, or function, of the product—including antigen uptake, processing and presentation, and subsequent immune response (Keilholz et al., J. 2002. J. Immunother. 25:97-138; Hinz et al., 2006. J Immunother. 29:472-476). For CTL-inducing immunotherapies, assays measuring cell proliferation, cell lysis or cytokine production (Whiteside and Gooding, 2003. 31:63-71; Keilholz et al., 2006. Clin. Cancer Res. 12:2356s-2352s; Davis et al., 2003; Whelan et al., 2006. Personalized Med. 3:79-88) are presently viewed as the gold standard in spite of their semi-qualitative nature (Hinz et al., 2006; Keilholz et al., 2006). These assays rely on inherently empirical biological materials, including T-cell clones or human peripheral blood lymphocyte populations, to produce quantitative, precise and reproducible results when the condition of the cell culture, other biological samples, instruments and users can differ between applications (Copier, et al., 2007. Vaccine 25S:B35-46). Active immunotherapies that target T-cell anergy, prove even more difficult to assess due to the difficulty to replicate surrogates of this activity in vitro. The reliance on cell-based reagents, with their inherent drift in properties, typical reduction in activity due to extended storage conditions and experience of assay bias complicates quality assurance efforts in assay standardization (Mosca et al., 2001. Surgery 129:248-254; Hinz et al., 2006). These current shortcomings encourage the development of new methods providing a quantitative measure of potency for both defined antigen and mixed antigen vaccines.

Active immunotherapies rely on the activities of the HLA class I and class II, and cognate interactions with T-cell receptors expressed on the surface of scanning T-lymphocytes. HLA class I is expressed on the surface of all nucleated human cells and, via its display of restricted peptide processed from intracellular proteins, presents a regular snapshot of the expressed proteins within a cell—acting like a proteomic biomarker chip for cellular status and antigen processing.

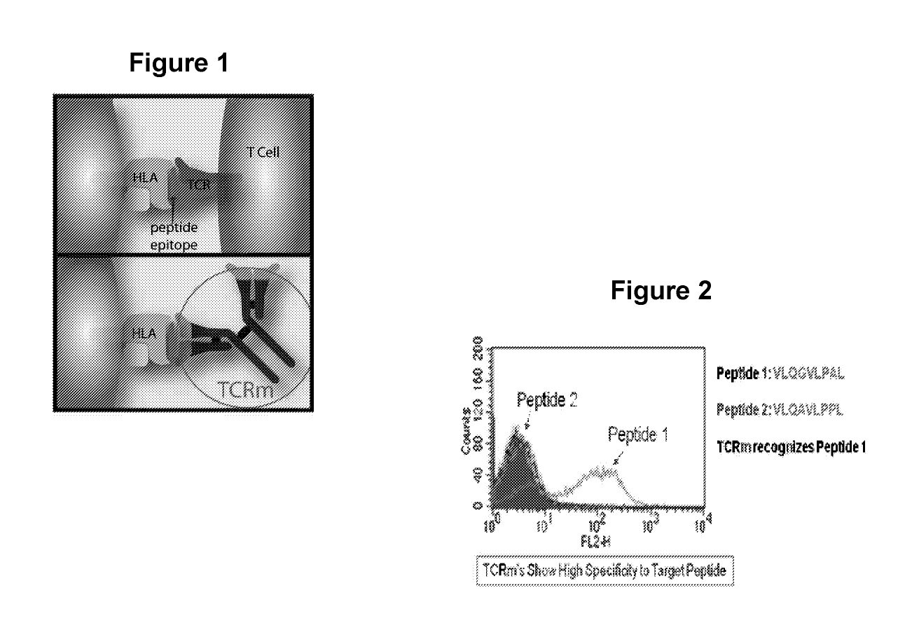

The interaction between the T-cell receptor and the peptide-HLA complex is central to the adaptive immune response—however its complicated nature presents particular challenges for integration into medical diagnosis and therapy. The inventors have previously demonstrated, in the parent applications referenced herein above and incorporated herein by reference, the development of a new type of monoclonal antibody (MAb), known as a T-Cell Receptor mimic (TCRm) that recognizes specific peptide-HLA complexes (FIG. 1). These TCRm antibodies have specific detection abilities at concentrations <150 pM, similar to the high avidity CTL lines classically used in binding assays (Wittman et al., 2006. J. Immunol. 177:4187-4195; Weidanz et al., J. Immunol. 2006. 177: 5088-5097; Weidanz et al., J Immunol Methods. 318:47-58; Neethling et al., 2008 Vaccine. February 25 epub). The impressive specificity of TCRm antibodies coupled with their ability to recognize validated disease biomarkers in the form of particular peptide-HLA complexes demonstrates that they represent new tools to augment and/or replace T-cell based assays (Neethling et al., 2008; Kageyama et al., 1995 J. Immunol. 154:567-576; Yang et al., 2002. J. Immunol 169:531-539).

TCRm antibodies show high affinity to the particular restricted peptide displayed in the context of the cognate HLA molecule used to produce the antibody. FIG. 2 shows an example of the specificity where a TCRm was raised against Peptide 1 and is unable to recognize (as displayed via a flow cytometry staining assay) Peptide 2, which differs from Peptide 1 in only two of the nine amino acid positions.

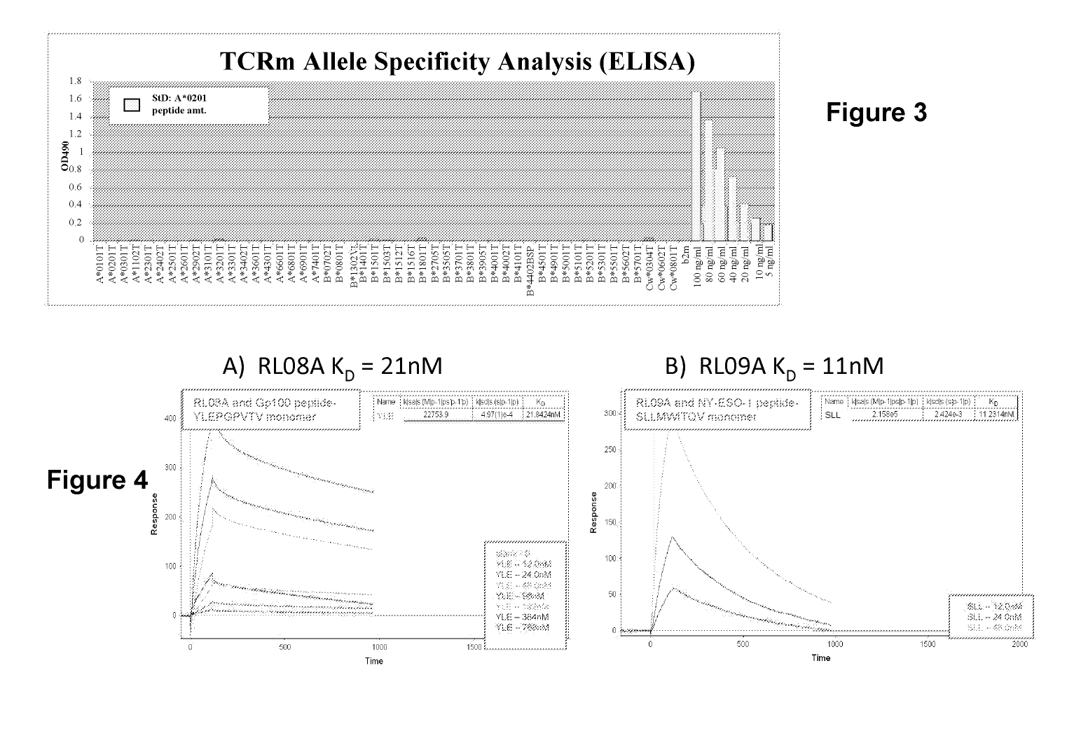

TCRm antibodies have expected properties of monoclonal antibodies. They have high binding specificity to very specific peptide-HLA complexes and as demonstrated in FIG. 3, do not cross react with non-target HLA.

And as demonstrated in FIG. 4, TCRm's have binding affinities that are similar to that of the T-cell receptor with Kd values of many TCRm antibodies <25 nM as determined by peptide titration and plasmon resonance experiments.

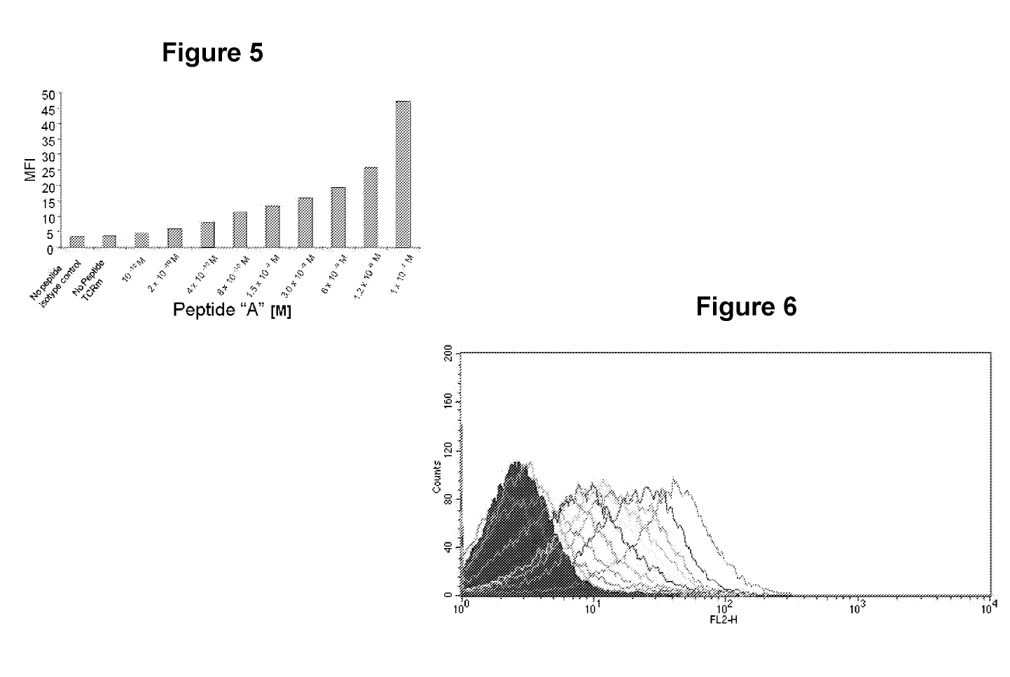

TCRm antibodies also show dramatic dynamic range with regards to sensitivity, where T2 cells pulsed with picomolar concentrations of peptides can be readily identified by the appropriate TCRm antibody (FIGS. 5 and 6). These data establish that TCRm antibodies have all the desired properties of monoclonal antibodies widely used in various quality control assays for biologic products.

However, the inventive concept(s) is to be understood to not be limited to the use of TCRm's. In addition to TCRm's, any agent capable of directly detecting peptide/MHC complexes on the surface of a cell and are capable of quantitatively measuring the number of peptide/MHC complexes present on the surface of a cell through a binding event may be utilized in accordance with the presently disclosed and claimed inventive concept(s). Examples of particular agents that may be utilized include, but are not limited to, soluble T-cell receptors, extracted T-cell receptors, antibodies, antibody fragments and the technologies described in any of the following US patents/publications: US Publication No. US 2006/0115470 A1, published on Jun. 1, 2006 and filed by Silence et al., on Nov. 7, 2003; US Publication No. US 2007/0178082 A1, published on Aug. 2, 2007 and filed by Silence et al., on Nov. 7, 2003; US Publication No. US 2006/0246477 A1, published on Nov. 2, 2006 and filed by Hermans et al., on Jan. 31, 2006; US Publication No. US 2006/0211088 A1, published on Sep. 21, 2006, and filed by Hermans et al., on Mar. 13, 2006; US Publication No. US 2005/0214857 A1, published on Sep. 29, 2005, and filed by Lasters et al., on Dec. 11, 2002; U.S. Pat. No. 6,818,418, issued to Lipovsek et al., on Nov. 16, 2004; U.S. Pat. No. 7,115,396, issued to Lipovsek et al., on Oct. 3, 2006; US Publication No. US 2005/0255548 A1, published on Nov. 17, 2005 and filed by Lipovsek et al., on Nov. 15, 2004; US Publication No. US 2007/0082365 A1, published on Apr. 12, 2007 and filed by Lipovsek et al., on Oct. 3, 2006; US Publication No. US 2006/0246059 A1, published on Nov. 2, 2006 and filed by Lipovsek et al., on Jul. 7, 2006; US Publication No. US 2006/0270604 A1, published on Nov. 30, 2006 and filed by Lipovsek et al., on Jul. 7, 2006; US Publication No. US 2008/0139791 A1, published on Jun. 12, 2008 and filed by Lipovsek et al., on Jun. 12, 2008; US Publication No. US 2006/0286603 A1, published on Dec. 21, 2006 and filed by Kolkman et al., on Mar. 28, 2006; US Publication No. US 2005/0053973 A1, published on Mar. 10, 2005 and filed by Kolkman et al, on May 5, 2004; US Publication No. US 2005/0089932 A1, published on Apr. 28, 2005 and filed by Kolkman et al., on Jun. 17, 2004; US Publication No. US 2004/0175756 A1, published on Sep. 9, 2004 and filed by Kolkman et al., on Oct. 24, 2003; US Publication No. US 2005/0048512 A1, published on Mar. 3, 2005 and filed by Kolkman et al., on Oct. 24, 2003; US Publication No. US 2005/0221384 A1, published on Oct. 6, 2005 and filed by Kolkman et al., on Oct. 15, 2004; US Publication No. US 2006/0223114 A1, published on Oct. 5, 2006 and filed by Stemmer et al., on Nov. 16, 2005; US Publication No. US 2006/0234299 A1, published on Oct. 19, 2006 and filed by Stemmer et al., on Nov. 16, 2005; US Publication No. US 2008/0003611 A1, published on Jan. 3, 2008 and filed by Silverman et al., on Jul. 12, 2006; US Publication No. US 2006/0286066 A1, published on Dec. 21, 2006 and filed by Basran on Dec. 22, 2005; US Publication No. US 2006/0257406 A1, published on Nov. 16, 2006 and filed by Winter et al., on May 31, 2005; US Publication No. US 2006/0106203 A1, published on May 18, 2006 and filed by Winter et al., on Dec. 28, 2004; US Patent No. US 2006/0263768 A1, published on Nov. 23, 2006 and filed by Tomlinson et al, on Apr. 28, 2006; US Publication No. 2007/0065440 A1, published on Mar. 22, 2007 and filed by Tomlinson et al., on Apr. 10, 2006; U.S. Pat. No. 6,696,245, issued to Winter et al., on Feb. 24, 2004; US Publication No. US 2006/0280734 A1, published on Dec. 14, 2006 and filed by Winter et al., on Jun. 24, 2005; US Publication No. US 2006/0083747 A1, published on Apr. 20, 2006 and filed by Winter et al., on Jun. 24, 2005; US Publication No. US 2004/0202995 A1, published on Oct. 14, 2004 and filed by de Wildt et al., on Apr. 9, 2003; U.S. Pat. No. 7,235,641, issued Jun. 26, 2007 to Kufer et al.; US Publication No. US 2003/0148463 A1, published on Apr. 7, 2003 and filed by Kufer et al., on Dec. 19, 2002; U.S. Pat. No. 7,227,002, issued to Kufer et al., on Jun. 5, 2007; U.S. Pat. No. 7,323,440, issued to Zoeher et al., on Feb. 12, 2003; U.S. Pat. No. 6,723,538, issued to Mack et al., on Apr. 20, 2004; U.S. Pat. No. 7,112,324, issued to Dorken et al., on Sep. 26, 2006; U.S. Pat. No. 7,250,297, issued to Beste et al., on Jul. 31, 2007; U.S. Pat. No. 6,849,259, issued to Haurum et al., on Feb. 1, 2005; US Publication No. 2008/0131882 A1, published on Jun. 5, 2008 and filed by Rasmussen et al., on Jul. 20, 2005; U.S. Pat. No. 5,670,626, issued to Chang on Sep. 23, 1997; U.S. Pat. No. 5,872,222, and issued to Chang on Feb. 16, 1999. The contents of each of the above-referenced patents and patent applications are hereby expressly incorporated herein by reference in their entirety.

Other Examples of particular agents that may be utilized in accordance with the presently disclosed and claimed inventive concept(s) are described in detail in parent application U.S. Ser. No. 61/191/871, filed Sep. 12, 2008, the entire contents of which has been previously incorporated herein by reference.

In the Example described herein after, TCRm monoclonal antibodies are utilized to directly detect a relative density of processed peptide-epitopes presented on the surface of vaccine-treated mDCs. The TCRm antibodies generated recognize specific peptide-HLAA2 epitopes derived from the hCGβ antigen. The vaccine is an antibody-antigen fusion protein developed at Celldex Therapeutics that specifically targets the mannose receptor on DCs and upon binding initiates rapid vaccine internalization (Ramakrishna et al., 2004). The processing and presentation of the antigen in the vaccine was enabled by further treatment with an adjuvant such as Poly I:C and confirmed using peptide-specific T cell lines. The presently disclosed and claimed inventive concept(s) demonstrates that the TCRm antibody was useful in corroborating the observed CTL activity by: (1) specifically inhibiting T cell stimulation, and (2) detection of HLA-A2-TMT and HLA-A2-GVL peptide complexes in vaccine-treated mDCs. Thus, the presently disclosed and claimed inventive concept(s) enables the use of agents, such as but not limited to TCRm mAbs, for the detection and quantitation of a relative density of specific peptide-HLA class I complexes on vaccine-treated mDCs and represents an important tool to measure the potency of CTL-inducing vaccines.

The presently disclosed and claimed inventive concept(s) is related to methods of assaying vaccine potency. The “potency of a vaccine composition” is defined as a predefined minimum level of potential biological activity, such as but not limited to, stimulation of antigen-specific CTL lines or clones. It has been shown that a density of specific peptides displayed by MHC class I complexes directly correlates with the CTL response to virus and cancer, and therefore the presently disclosed and claimed inventive concept(s) is related to the use of antibodies specific for peptide-MHC class I complexes to measure the potency of CTL-inducing vaccines. The measurement of peptide-MHC class I complexes can be quantitatively determined using the methods described using TCRm antibodies. Said quantitative measurement may be related to a relative number of peptide/MHC complexes per cell, or may be related to an actual number of peptide/MHC complexes per cell.

In one embodiment, the methods utilize a T-cell receptor mimic, as described in detail hereinabove and in U.S. Ser. No. 11/809,895, filed Jun. 1, 2007, and in US published applications US 2006/0034850, filed May 27, 2005, and US 2007/00992530, filed Sep. 7, 2006, which have previously been incorporated herein by reference. The T-cell receptor mimic utilized in the methods of the presently disclosed and claimed inventive concept(s) comprises an antibody or antibody fragment reactive against a specific peptide/MHC complex, wherein the antibody or antibody fragment can differentiate the specific peptide/MHC complex from the MHC molecule alone, the specific peptide alone, and a complex of MHC and an irrelevant peptide. The T cell receptor mimic may be produced by any of the methods described in detail in the patent applications listed herein above and incorporated herein; briefly, the T cell receptor mimic is produced by immunizing a host with an effective amount of an immunogen comprising a multimer of two or more specific peptide/MHC complexes.

The T cell receptor mimic utilized in accordance with the presently disclosed and claimed inventive concept(s) may be produced by a method that includes identifying a peptide of interest, wherein the peptide of interest is capable of being presented by an MHC molecule, and wherein the vaccine composition comprises the peptide of interest. An immunogen comprising a multimer of two or more peptide/MHC complexes is then formed, wherein the peptide of the peptide/MHC complex is the peptide of interest. An effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced, wherein the desired antibodies can differentiate the peptide/MHC complex from the MHC molecule alone, the peptide of interest alone, and a complex of MHC and irrelevant peptide. The desired antibodies are then isolated.

Table I provides a list of some of the peptides that have been utilized to produce TCRm's by the methods described in detail in U.S. Ser. No. 11/809,895, filed Jun. 1, 2007, and in US published applications US 2006/0034850, filed May 27, 2005, and US 2007/00992530, filed Sep. 7, 2006, which have previously been incorporated herein by reference. The use of TCRm's produced using any of the peptides of SEQ ID NOS:1-81 is specifically contemplated by the presently disclosed and claimed inventive concept(s). However, it is to be understood that the presently disclosed and claimed inventive concept(s) is not limited to TCRm's produced using said peptides, but rather the scope of the presently disclosed and claimed inventive concept(s) encompasses TCRm's raised against any specific peptide/MHC complex.

TABLE I
Peptides Utilized in the Methods of
U.S. Ser. Nos. 11/140,644,
11/517,516; and 11/809,895

| | SEQ | |
|---|---|---|
| | ID | |
| Sequence | NO: | Origin |
| LLGRNSFEV | 8 | Tumor suppressor |
| | | p53 (264-272) |
| VLMTEDIKL | 9 | eukaryotic |
| | | transcription |
| | | initiation factor 4 |
| | | gamma (720-728) |
| KIFGSLAFL | 5 | tyrosine kinase-type |
| | | cell surface receptor |
| | | Her2 (EC 2.7.1.112) |
| | | (C-erbB-2) (369-377) |
| TMTRVLQGV | 2 | human chorionic |
| | | gonadotropin-β (40- |
| | | 48) |
| VLQGVLPAL | 3 | human chorionic |
| | | gonadotropin-β (44- |
| | | 53) |
| GVLPALPQV | 4 | human chorionic |
| | | gonadotropin-β (47- |
| | | 55) |
| YLLPAIVHI | 10 | p68 |
| TLAYLIFCL | 11 | CD 19 (296-304) |
| YLEPGPVT | 12 | GP100 (280-288) |
| SLLMWITQV | 13 | NY-ESO-1 (157-165) |
| ILAKFLHWL | 14 | Human telomerase |
| | | reverse transcriptase |
| | | (hTERT) (540-548) |
| GPRTAALGLL | 7 | Reticulocalbin |
| EVDPIGHLY | 6 | Mage-3 |
| AAGIGILTV | 15 | MART-1 (26-35) wild |
| | | type |
| AIMDKNIIL | 16 | |
| ALGIGILTV | 17 | MART-1 (26-35)(27L) |
| ALMPVLNQV | 18 | MTR3 |
| ATDFKFAMY | 19 | G1/S-specific cyclin- |
| | | D2 |
| ATTNILEHY | 20 | TRP-2-6b |
| AVLPPLPQV | 21 | bLH (67-75) |
| EADPTGHSY | 22 | Mage-1 |
| ELTLGEFLKL | 23 | Survivin |
| FLAEDALIITV | 24 | H-RYK |
| FLSTLTIDGV | 25 | HLA-A*0201-RE from |
| | | endothelium |
| FLSELTQQL | 26 | MIF |
| FLYDDNQRV | 27 | Topoisomerase |
| GILGFVFTL | 28 | Influenza MI |
| GLNEEIARV | 29 | HEC1 |
| GVLPNIQAV | 30 | |
| GVYDGEEHSV | 31 | Mage-B2 |
| IADMGHLKY | 32 | Proliferating cell |
| | | nuclear antigen |
| ILDQKINEV | 33 | ODC1 |
| ILKEPVHGV | 34 | HIV reverse |
| | | transcriptase |
| ILNSRPPSV-OH | 35 | Modified |
| IMDQVPFSV | 36 | Gp100 (208-217) |
| | | (2M) |
| IPSIQSRGL | 37 | |
| ITDQVPFSV | 38 | Gp100 (209-217) wild |
| | | type |
| ITNSRPPSV- | 39 | Native (wild type) |
| OH | | |
| KIFGALAFL | 40 | S5A |
| KIFGGLAFL | 41 | S5G |
| KIFGKLAFL | 42 | S5K |
| KIGEGTYGV | 43 | CK2 |
| KKLLTQHFVQE | 44 | Mage-3 (157-170) |
| NYLEY | | |
| KLGEGTYGV | 45 | |
| KLMSPKLYV | 46 | 19-(150-158) |
| KLQELNYNL | 47 | Stat1 |
| KVLEYVIKV | 48 | Mage-1 (278-286) |
| LKMESLNFI | 49 | 20-(147-155) |
| LPFDRTTVM | 50 | INF B7-2 |
| NAITNAKII | 51 | RSV M |
| NLVPMVATV | 52 | CMV pp65 |
| QPEWFRNIL | 53 | |
| QPEWFRNVL | 54 | |
| RMFPNAPYL | 55 | Wilm's tumor gene |
| | | WT1 (126-134) |
| RPYSNVSNL | 56 | B7B2, set-binding |
| | | factor 1 |
| SIGGVFTSV | 57 | S(I)G9 |
| SLFLGILSV | 58 | 20-(188-196) |
| SLLMWITQC | 59 | HLA-A*0201-RE NY- |
| | | ESO-1 WT (157-165) |
| SLLEKREKT | 60 | HLA-A*0201-RE from |
| | | SP-17- |
| STAPPAHGV | 61 | MUC1 |
| STPPPGTRV | 62 | HLA-A*0201-RE from |
| | | p53 (149)- |
| SVGGVFTSV | 63 | SVG9 |
| SYIGSINNI | 64 | HRSV M2-1 |
| TLHEYMLDL | 65 | HPV16 E7-1 |
| TLQDIVLHL | 66 | HPV18 E7-1 |
| TMMRVLQAV | 67 | bLH (60-68) |
| TPQSNRPVM | 68 | B7A9, RNA pol II |
| | | polypeptide A |
| VLQAVLPPL | 69 | bLH(64-72)- |
| VLQELNVTV | 70 | PR-1 (169-177) |
| VMAGVGSPYV | 71 | Her2-(773-782)- |
| YIFGSLAFL | 72 | |
| YKYKVVKIEPLG | 73 | P46, 13 mer, HIV-1 |
| V | | envelope |
| YLEPGPVTA | 74 | Gp100: 280-288 Wild |
| | | type |
| YLEPGPVTV | 75 | Gp10: 280-288 (288V) |
| YLLEMLWRL | 76 | Epstein-Barr virus (EBV) |
| YMLDLQPETT | 77 | HPV16 (E7₁₁₋₂₀) |
| RLDDDGNFQL | 78 | West Nile Virus NS2b |
| ATWAENIQV | 79 | West Nile Virus peptide |
| | | ATW9-WNV |
| YTMDGEYRL | 80 | West Nile Virus NS3 |
| | | YL9 |
| SLTSINVQA | 81 | West Nile Virus peptide |
| | | NS4b SA9 |

The agents, such as but not limited to, T cell receptor mimics, described and claimed herein are capable of directly detecting low densities of specific MHC-peptide complexes present on the surface of cells, such as tumor or infected cells. In this fashion, the agents, such as but not limited to, T cell receptor mimics, can thereby be utilized to detect the presence of specific peptide/MHC complexes present on the surface of cells treated with a vaccine, wherein the peptide of the specific peptide/MHC complex is a product of the degradation of a vaccine (or, the vaccine itself, when the vaccine is directly delivered in peptide form).

When a T cell receptor mimic is utilized as the agent, T cell receptor mimic may have a binding affinity for the specific peptide/MHC complex of about 10 nanomolar or greater.

The agent utilized in accordance with the presently disclosed and claimed inventive concept(s) may be provided with a detection moiety bound thereto to aid in measuring the level of specific peptide/MHC complex present on the surface of the antigen presenting cell. Any detection moiety known in the art or otherwise contemplated by a person having ordinary skill in the art for use with the presently disclosed and claimed inventive concept(s) is encompassed by the scope of the presently disclosed and claimed inventive concept(s). Particular non-limiting examples of detection moieties that may be utilized in accordance with the presently disclosed and claimed inventive concept(s) have been described in detail herein above.

The methods of the presently disclosed and claimed inventive concept(s) include the step of providing a vaccine composition and delivering the vaccine composition to at least one antigen presenting cell to provide a vaccine-treated cell. The vaccine composition may be provided in any form known in the art; for example but not by way of limitation, the vaccine composition may be directly provided as at least one protein/polypeptide that may be processed into peptides by the antigen presenting cell. Alternatively, the vaccine composition may be provided in the form of a nucleic acid segment encoding the at least one protein/polypeptide, wherein the antigen presenting cell expresses the nucleic acid segment and produces the protein/polypeptide encoded by the nucleic acid segment. In yet another embodiment, the vaccine composition may be provided in the form of a specific peptide known to be an epitope expressed in the context of MHC molecules. In a further embodiment, the vaccine composition may be a nucleic acid segment encoding such peptide epitope (wherein the antigen presenting cell expresses said nucleic acid segment and produces said peptide epitope).

The antigen presenting cell to which the vaccine composition is delivered may be any cell that is capable of presenting peptides in the context of MHC molecules. When the vaccine composition is presented in the form of a protein/polypeptide (or a nucleic acid segment encoding same), the antigen presenting cell must also be capable of processing proteins/polypeptides into peptides that may be presented in the context of MHC molecules. Examples of antigen presenting cells that may be utilized in accordance with the presently disclosed and claimed inventive concept(s) include, but are not limited to, dendritic cells, macrophages, B cells and combinations thereof.

Once the vaccine-treated cell is produced, it is reacted with the agent, such as but not limited to the T cell receptor mimic, whereby the agent binds to the cell surface if the specific peptide/MHC complex utilized to produce the agent is present on the cell surface.

The number of specific peptide/MHC complexes present on the surface of the vaccine-treated antigen presenting cell are then quantitatively measured; said methods of quantitative measurement may include both relative quantitation based on delta MFI (ΔMFI) values as well as absolute complex number determinations. Methods of quantitatively measuring the number of specific peptide/MHC complexes include, but are not limited to, correlating TCRm binding ΔMFI values derived from flow cytometry with appropriate standard, where a known quantity of the staining reagent, such as but not limited to PE, APC or other materials, is present on a number of standards that allow separation via flow cytometry, ΔMFI determination and linear regression formula determination. ΔMFI values of unknown samples can be measured by flow cytometry, and quantitative differences can be determined based on relative number of peptide-MHC complexes. For a non-relative determination, unknown samples are analyzed, such as by TCRm staining, and the ΔMFI values are compared with the linear regression formula to determine the numbers of staining reagent present. The number of staining reagent present on the antibody measured with flow is then used to determine the average number of peptide-MHC molecules present per cell in an assay.

The terms “quantitative measurement” and “quantitatively measuring” as used herein will be understood to refer to establishing a differential value related to the number of peptide-MHC complexes present on the surface of vaccine treated cells by relative means, such as but not limited to, by using ΔMFI values (which directly correlates with the number of peptide-MHC complexes) or a process to convert these relative values into absolute numbers of peptide-MHC complexes as described above.

The potency of the vaccine is then determined, based on the quantitative measurement of the number of specific peptide/MHC complex present on the surface of the vaccine-treated antigen presenting cell.

Potency is measured by comparing the threshold amount or activity of the vaccine to induce a T-cell response, such as but not limited to a CTL response or T-cell anergy, such that it is meaningful to a biological effect in vivo. In this manner, the T cell receptor mimic binding assay determines the correlative density of the HLA-peptide complexes on the antigen presenting cell.

Examples are provided hereinbelow. However, the presently disclosed and claimed inventive concept(s) is to be understood to not be limited in its application to the specific experimentation, results and laboratory procedures. Rather, the Examples are simply provided as various embodiments and are meant to be exemplary, not exhaustive.

### Example 1

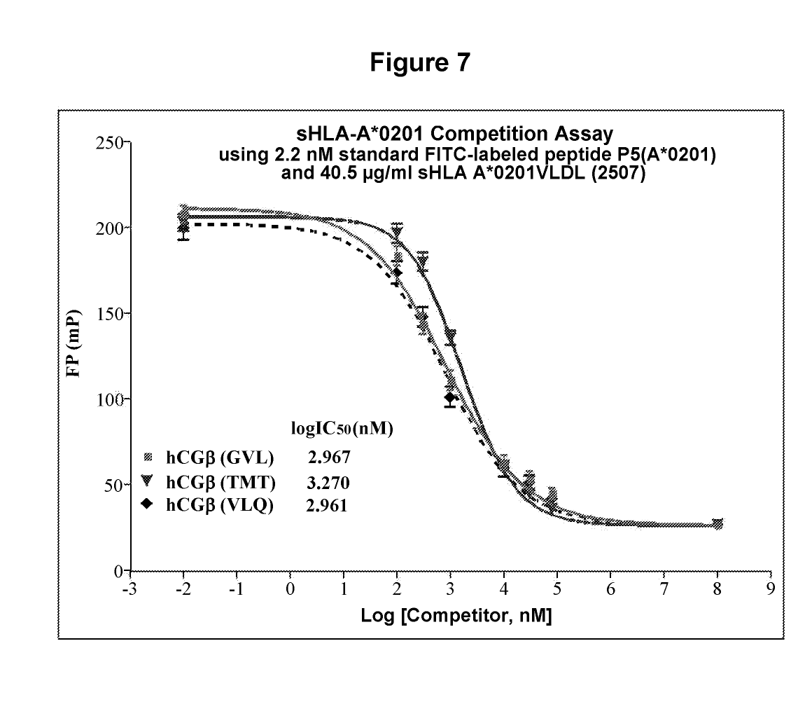

Validation of previously identified hCGβ peptide epitopes by PolyTest. A major parameter determining cell-surface presentation of a given peptide is the affinity of the peptide for HLA class I molecules. In this regard, several lines of evidence, both at the biological and functional level, emphasize the choice of high affinity peptides in TCR mimic generation. Epitopes need to be selected that have the requisite binding affinity established to be successful. Our standardized PolyTest approach (Buchli et al., 2005; Buchli et al., 2006; and Buchli et al., 2004) is used in the determination of the inhibitory concentration (IC₅₀) on positively identified peptide candidates. The method is quantitative and yields affinity values with a high degree of accuracy for each of the three peptides used in this example. Recent results published by Dangles et al. (2002) indicated that the TAA hCGβ possesses numerous antigenic determinants able to stimulate CD8+ T lymphocytes. In addition, several hCGβ-derived peptides were found to exhibit HLA-A*0201 binding capabilities. Three of them, namely TMTRVLQGV (40-48; SEQ ID NO:2), VLQGVLPAL (44-52; SEQ ID NO:3) and GVLPALPQV (47-55; SEQ ID NO:4) seemed of high affinity able to stabilize HLA complexes on T2 cell surfaces (Table II). These peptides were reevaluated using PolyTest to obtain more accurate and quantitative affinity values. Results seen in FIG. 7 demonstrate that the three peptides express similar binding affinities in a close log IC₅₀ range between 2.9 and 3.2, indicating each peptide has high affinity for HLA-A*0201.

TABLE II
Overlapping Peptides from hcGβ with
Similar Binding Affinity for HLA-A*0201

| Antigen | Location | Designation | Sequence | SEQ ID NO: |
|---|---|---|---|---|
| hCGβ | 40-48 | TMT | TMTRVLQGV | 2 |
| hCGβ | 44-52 | VLQ | VLQGVLPAL | 3 |
| hCGβ | 47-55 | GVL | GVLPALPQV | 4 |

Generation of TCRm's, characterization of binding to specific peptide, and demonstration of target display on tumor cells. Following the synthesis of HLA-A2 tetramers loaded with peptide (TMT or GVL), splenocytes isolated from immunized mice were prepared for fusion with the P3X-63Ag8.653 myeloma cell line and plated in a semi-soft cellulose medium. After about two weeks, colonies were identified, picked to individual wells of a 96 well plate for expansion and the hybridoma supernatants were screened for reactive antibodies. Table III shows the results from hybridoma fusions for each peptide-HLA-A2 immunogen. Several IgG1, IgG2a and IgG2b antibodies were selected from each immunization group.

TABLE III
Total Hybridoma Clones Screened and Number of Positive
(Antibody Reacted with Specific Peptide HLA-A2
Complex) Clones Isolated

| Immunogen | TMT-HLA-A2 | GVL-HLA-A2 |
|---|---|---|
| Number of Clones | 850 | 1980 |
| Number of TCRmimics | 15 | 28 |

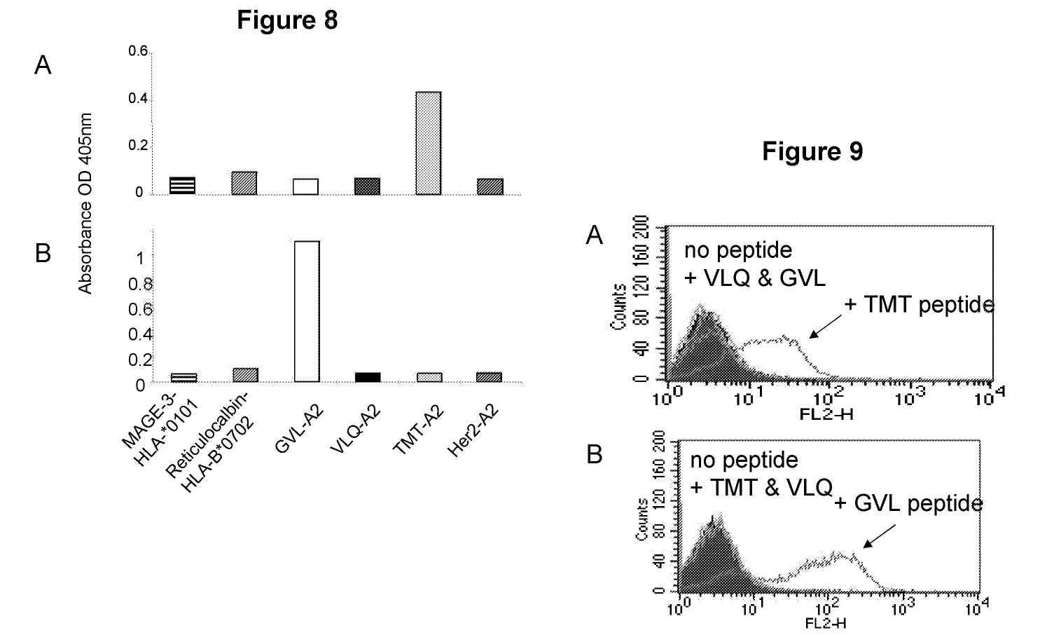

To determine the peptide-specific reactivity of 3F9 (anti-TMT-A2) and 1810 (anti-GVL-A2), the mAbs were first purified by affinity chromatography on a protein-G column and their binding specificity assessed by ELISA. Each antibody (tested at 1 μg/ml) showed significant reactivity for its respective peptide without any detection of binding to the irrelevant peptides (FIG. 8). These findings suggest that each of the antibodies selected has no detectable crossreactivity with either the HLA complex or any of a series of HLA complexes loaded with various peptides, which also bind HLA-A2.

Although each TCRms recognize its cognate peptide-A2 target in coated wells, it was unclear whether these mAbs would recognize the specific peptide when loaded into HLAA*0201 complexes expressed on a cell surface. In order to ensure that these TCRms recognize their specific peptide in the context of the native HLA-A2, their binding to T2 cells pulsed with 20 μM of specific, irrelevant peptides or no peptide was analyzed. FIG. 9 shows that both TCRms stain T2 cells pulsed with only specific peptide. These results confirm the fine and unique specificity of each TCRmimic for their respective peptide present in the HLA-A2 complex.

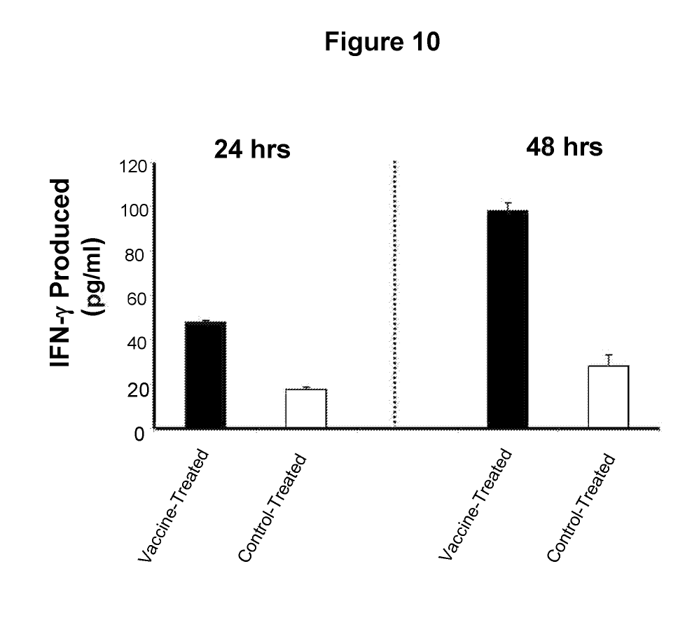

Vaccine-treated DCs elicit Ag-specific CTL response. To assess anti-hCGβ specificity of the CTL line, DCs were treated for 3 days with either the B11-hCGβ vaccine or the B11-CEA control vaccine to target DCs for 3 days and then matured for 24 h using Poly I:C. The CTL line was then incubated with vaccine or vehicle-treated DCs at a ratio of 1:1 for 24 and 48 h. CTL reactivity was measured by sampling culture supernatant for IFN-γ production. As seen in FIG. 10, the IFN-γ response was significantly higher for CTL incubated for 24 h with DC treated with the B11-hCGβ vaccine (50 pg/ml) than with control treated DCs (15 pg/ml). CTL stimulation for 48 h resulted in even a greater difference in IFN-γ levels between vaccine-treated and vehicle-treated DC, indicating an hCGβ-specific CTL response for peptide epitopes presented on 3 day vaccine-treated DCs.

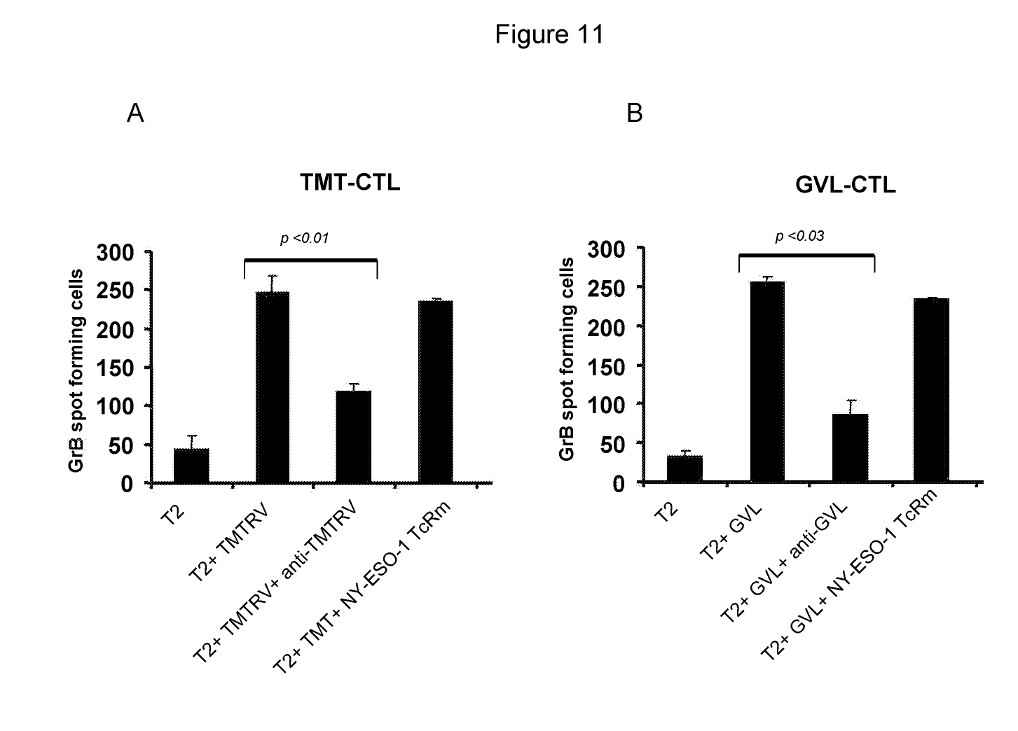

Inhibition of CTL stimulation with peptide-epitope specific TCRm CTL lines were generated against the TMT and GVL peptide-HLA-A*0201 epitopes using autologous dendritic cells. CTL peptide specificity was determined using T2 cells alone or T2 cells pulsed with relevant peptide. As shown in FIG. 11, TMT and GVL peptide-specific CTL lines responded to T2 cells presenting relevant peptide but not to T2 cells alone. Further, granzyme-B production by CTL lines specific for TMT and GVL peptide-epitopes was inhibited by the addition of anti-TMT and anti-GVL TCRm, respectively. In this example, peptide-epitope specific TCRm were used to confirm CTL recognition specificity for the TMT peptide and GVL peptide epitopes.

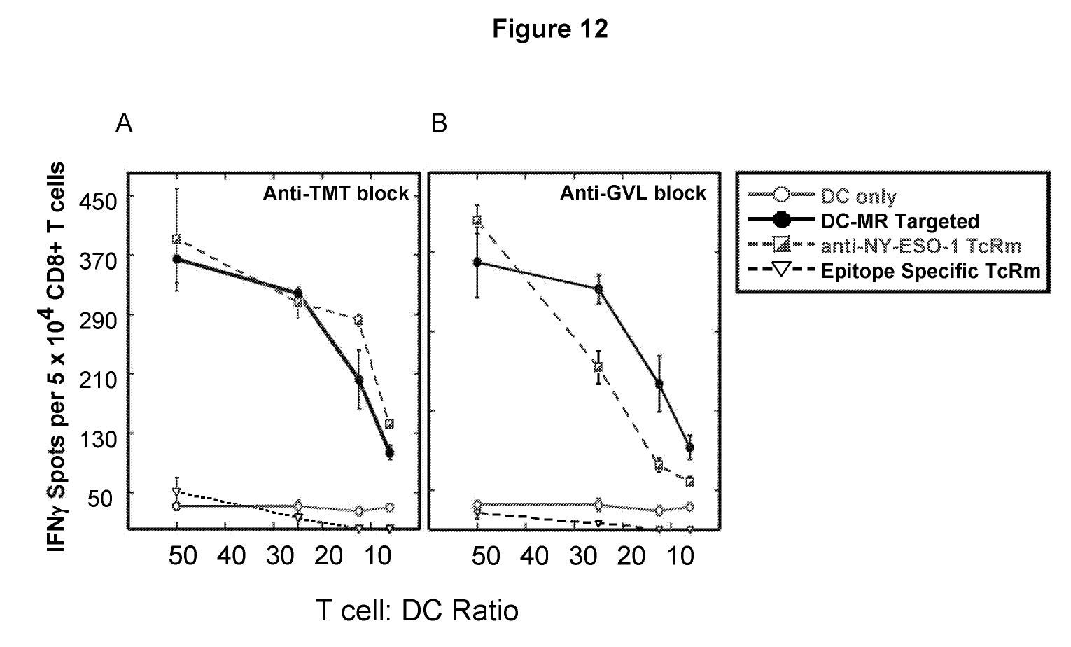

Peptide-specific CTL recognize TMT and GVL peptide-HLA-A2 complexes on vaccine-treated autologous DCs. To this point it has been shown that vaccine-targeted DC could stimulate anti-hCGβ CTL, indicating that the DCs were processing and presenting peptides from the hCGβ vaccine construct. To determine whether the TMT and/or GVL peptides were endogenously processed and presented, autologous DCs were treated with the B11-hCGβ vaccine conjugate and CTL were assessed for IFN-γ production. As shown in FIG. 12, the CTL response was specific for TMT peptide and GVL peptide epitopes and directly correlated with effector cell to target cell ratio (E:T). Furthermore, the response was inhibited using the respected TMT or GVL peptide-epitope specific TCRm but not with control TCRm (anti-NY-ESO-1 (157-165)-HLA-A2 TCRm). These findings indicate that TMT and GVL peptides are processed and presented in the context of HLA-A*0201 in vaccine-treated DCs and that TCRm antibodies are useful agents in validating the recognition specificity of the CTL response.

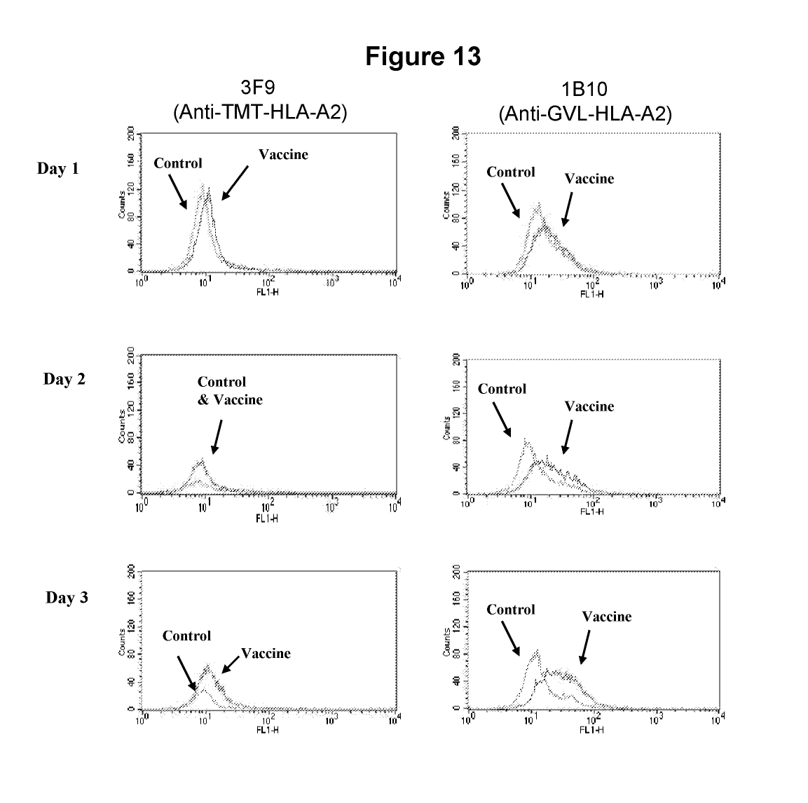

TCRm antibodies stain vaccine-treated dendritic cells. The use of TCRms to inhibit CTL response indicated indirectly the expression of specific peptide-epitope on the surface of DCs. Here the use of TCRm mAbs for direct validation of peptide-epitope expression on vaccine-treated DCs has been examined. First, the hypothesis that hCGβ peptides presented on the surface of vaccine treated DCs via HLA-A*0201 class I molecules are detectable using peptide-epitope specific TCRms was tested. Next, the kinetics of expression and the hierarchy of peptide presentation on the DCs was examined. Immature dendritic cells were treated with either vaccine or vehicle for up to 3 days and matured with Poly I:C at the different time points indicated. Using the anti-GVLpeptide-HLA-A2 TCRm (1B10) mAb, a dominant expression profile was detected for the GVL-peptide-epitope as early as 24 h. Interestingly, the intensity of the 1B10 TCRm staining signal increased at day 2 (MFI 28 versus 16 vehicle) and continued to increase (MFI 39 versus 19 vehicle) after 3 days of vaccine exposure (FIG. 13). In contrast, only a weak signal was observed on dendritic cells using the anti-TMT peptide-HLA-A2 TCRm (3F9) after 3 days of vaccine (FIG. 13). These findings raise interesting possibilities (α) permissiveness in processing and presentation of some (GVL) but not other (TMT) epitopes and (b) the kinetics of epitope generation may be different for different epitopes.

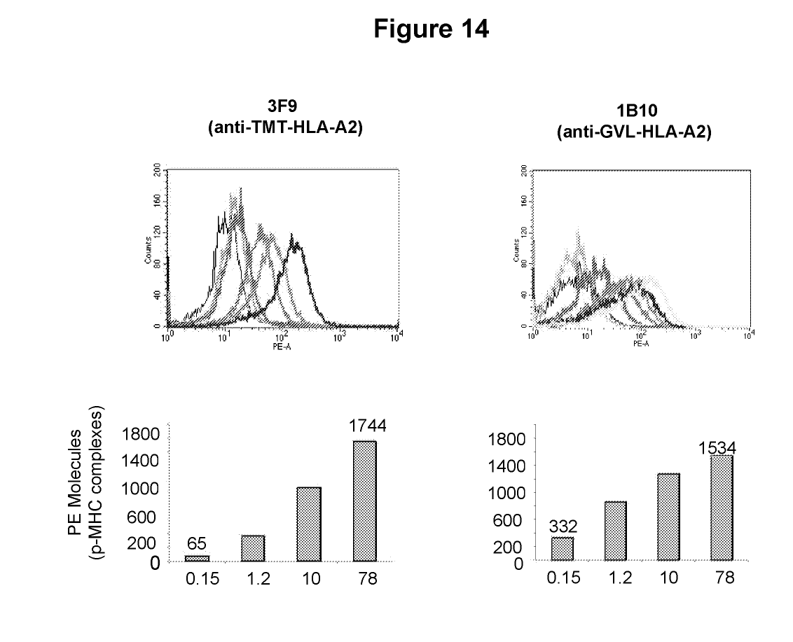

TCRm detection sensitivity. Next, the sensitivity of each antibody as a staining reagent was evaluated. This was done using flow cytometric analysis of T2 cells loaded with peptide ranging from 2000 nM down to 0.15 nM concentrations. Both TCRm clones (3F9 and 1B10) were able to stain T2 cells loaded with as little as 150 pM of peptide (FIG. 14). These findings indicate TCRm mAbs display detection sensitivity limits comparable to the lower detection limits reported for several high avidity CTL lines making TCRm antibodies highly sensitive tools for visualizing and quantitating specific peptide-MHC class I complexes on cells.

Discussion of Example 1

Dendritic cells are potent activators of CD4+ and CD8+ T cells and anti-tumor responses and have been extensively examined as a potentially useful immunotherapeutic approach for cancer treatment. This has led to the direct use of DCs as antigen delivery vehicles in a variety of experimental systems (Steinman, 1996; and Lou et al., 2004). The inventors and others have delivered antigens to DC by way of gene transduction (Chiriva-Internati et al., 2003) and via receptor-mediated endocytosis of whole proteins using receptor-specific antibodies (Ramakrishna et al., 2004; and He et al., 2004). In addition, mDCs have been successfully exploited as vehicles to deliver exogenously loaded synthetic peptides (Nakamura et al., 2005; and Godelaine et al., 2003). Specific targeting of vaccines to antigen-presenting cells such as DCs provides a model system for evaluating whether antigen processing has occurred and which immunogenic peptides have been presented by MHC molecules. However, current potency assays cannot directly measure specific peptide-MHC complexes. In this example, TCRm mAbs generated to two overlapping peptide-epitopes from the TAA hCGβ were used to directly show that presentation of both hCGβ-derived peptide-epitopes readily occurs on the surface of vaccine-treated DCs. Further, it was confirmed that the epitopes mapped by TCRm is identical to that seen by CTL. Most often MHC-peptide presentation is assessed by indirect means by monitoring a biological response of antigen-specific CTL to proliferate, mediate cell lysis or produce cytokines such as IL-2 and IFN-γ (Whiteside et al., 2003; and Gauduin, 2006). These responses, however, are not instantaneous, are labor and time intensive and are not quantitative (Petricciani et al., 2006). Further, these assays are impractical for evaluating potency of multiple batches of vaccines owing to the ephemeral nature of T cell-based reagents whose activity can fade with time (Petricciani et al., 2006). Therefore, the presently disclosed and claimed inventive concept(s) demonstrates that direct detection and quantitation of MHC-peptide complexes represent a novel surrogate marker for assessing CTL responses as was demonstrated in this example.

These findings are in line with the inventor's previous report wherein a Her2/neu (369)-HLA-A2-specific CTL line mediated lysis of target cells was dependent on the level of expression of Her2/peptide-HLA-A2 complexes on tumor cells (Weidanz et al., 2006). Still others have recently reported that a key variable that may be a determinant of T cell function is the density of epitope presented at the surface of APCs (Bullock et al., 2000; Wherry et al., 1999; Wherry et al., 2002; and Bullock et al., 2003).

TCRm antibodies can be used to directly detect and quantitate specific peptide-HLA class I epitopes on many cells including dendritic cells (Zehn et al., 2006; Zehn et al., 2004; and Kukutsch et al., 2000). The TCRm mAbs used in this example were found to exhibit unique binding specificity and exquisite detection sensitivity that was demonstrated by staining T2 cells pulsed with a low concentration of specific peptide (<150 pM). High avidity CTL lines reactive to TAA peptide-epitopes have been shown to have a lower detection limit in the 100 pM range (Kageyama et al., 1995; Yee et al., 1999; and Yang et al., 2002). A quantitative method using PE-labeled beads revealed that both the anti-TMT and anti-GVL TCRm mAbs recognized their cognate peptide-epitope at less than 60 peptide-epitope copies per cell. Thus, the TCRm mAbs and high avidity CTL lines have comparable detection sensitivity limits. The hCGβ tumor-associated antigen was selected because it is widely expressed by tumors of different histological origins and the B11-hCGβ antibody fusion vaccine has been previously shown to be internalized and capable of inducing CTL responses against the hCGβ peptide-epitopes including TMT peptide-HLA-A2 (He et al., 2004). He et al. reported that CTL generated using DC-treated with the B11-hCGβ vaccine lysed T2 cells pulsed with TMT peptide substantiating the immunogenicity of these two peptide epitopes. This model system allowed us to address two key points: (1) the question of whether each peptide epitope was presented by vaccine treated DCs and (2) the kinetics with which a particular peptide that was presented was indeed dominant. Future studies using our model system will address the hypothesis that the level of peptide-epitope expression is correlated with heightened CTL responses. One of the most intriguing aspects of the data at hand appears to be the kinetics of peptide-epitope presentation and the observation that the TMT and GVL peptide epitopes were detected as early as 24 h after vaccine treatment on the surface of DCs. Furthermore, the intensity of the anti-GVL peptide-HLA-A2 staining continued to increase reaching a maximum signal 72 h post vaccine-exposure of DCs. Our finding is in agreement with other studies (Bonifaz et al., 2002; and Yang et al., 2000) wherein immature DCs of mice were targeted via the DEC-205 receptor using an antibody coupled with OVA protein and followed the rate of antigen MHC presentation although neither study directly detected and quantitated specific peptide-epitope.

The methods of the presently disclosed and claimed inventive concept(s) allow for direct examination of the expression hierarchy of peptide-epitope presentation on vaccine-treated DCs. This has potential significance for vaccine design as many vaccines under development contain multiple peptide epitopes. A better understanding of the properties regulating peptide-epitope dominance could assist in developing more potent vaccines. Moreover, the ability to directly detect and quantitate peptide epitopes would potentially allow for screening of adjuvants and biological response modifiers that enhance the expression of a particular peptide-epitope of interest or even possibly modify peptide-epitope dominance.

Targeting specific peptide epitopes as surrogate markers for predicting a biological response was supported in this example. Previously, the inventors reported a direct correlation between Her2/neu (369) peptide-HLA-A2 epitope expression and CTL-mediated lysis of tumor cells (Weidanz et al., 2006). The presently disclosed and claimed inventive concept(s) further strengthens this concept using TCRm mAbs in assays not only to measure the potency of a manufactured vaccine lot but to also potentially be able to type tumor sections and DTH punch biopsies. In this regard, it will be curious to test the use of TCRm reagents for anomalies in tumor biomarker expression such as antigen loss variants (HLA, TAA, etc.). An important goal would be to determine whether HLA-A2 TCRms will clearly discriminate between intact HLA from those with structural mutations (polymorphisms) in the binding groove as also β2m loss variants.

Materials and Methods for Example 1

Antibodies and synthetic peptides. The conjugated polyclonal antibodies goat anti-mouse-IgG (H+L chains)-horseradish peroxidase (HRP) and goat antimouse IgG heavy chain-phycoerythrin (PE) were purchased from Caltag Biosciences (Burlingame, Calif.). The mouse IgG1 isotype control antibody was purchased from Southern Biotech (Birmingham, Ala.). Peptides TMTRVLQGV [residues 40-48, human chorionic gonadotropin-β peptide designated as TMT₍₄₀₎; SEQ ID NO:2], VLQGVLPAL [residues 44-52, human chorionic gonadotropin-β peptide, designated as VLQ₍₄₄₎; SEQ ID NO:3], GVLPALPQV [residues 47-55, human chorionic gonadotropin-β peptide, designated as GVL₍₄₇₎; SEQ ID NO:4], KIFGSLAFL [residues 369-377, Her2/neu peptide designated as Her2₍₃₆₉₎; SEQ ID NO:5], EVDPIGHLY [residues 161-169, MAGE-3 cancer testis antigen peptide designated as MAGE-1₍₁₆₁₎; SEQ ID NO:6], and GPRTAALGLL [residues 4-13, human reticulocalbin peptide, designated as Reticulocalbin; SEQ ID NO:7] were synthesized at the University of Oklahoma Health Sciences Center, Oklahoma City, Okla., using a solid-phase method and purified by HPLC to greater than 90%.

Cell lines. The human lymphoblastoid cell line T2 (HLA-A*0201) and the P3X-63Ag8.653 murine myeloma cell line used as a fusion partner were purchased from the American Type Culture Collection (ATCC, Manassas, Va.).

Generation of TCRm mAbs. Hybridomas producing the anti-TMT (designated 3F9) and anti-GVL (designated 1810) antibodies were made by Receptor Logic Ltd., as previously described in U.S. Ser. No. 11/809,895, filed Jun. 1, 2007, and in US published applications US 2006/0034850, filed May 27, 2005, and US 2007/00992530, filed Sep. 7, 2006 (all previously incorporated herein by reference). In addition, the control TCRm, anti-NY-ESO-1 (peptide 157-165)-HLAA*0201, was also produced by Receptor Logic. Briefly, mice (Balb/c) were repeatedly immunized with 50 μg of purified peptide-HLA-A*0201 complex and Quil-A adjuvant (Sigma, St. Louis, Mo.). After determining antibody reactivity against the immunogen, fusions were carried out using the Clonacell-HY Kit (Stem Cell Technologies, Vancouver, BC). Single clones were picked and screened for appropriate mAb production by ELISA (as described below); all three antibodies produced by the resulting hybridomas used in this study were IgG1 isotype. Large amounts of antibody-containing supernatant were generated and purified by affinity chromatography as previously described.

Fine specificity TCRm ELISA. Reactivity of purified TCRms was assessed by ELISA as previously described. Briefly, plates were coated overnight with purified complexes of HLA-A*0201-peptide, MAGE-3 peptide-HLA-A*0101 or Reticulocalbin peptide-HLA-B*0702 in PBS. After blocking with 5% milk, purified mAb was added to the plate and incubated for 2 h at room temperature (RT). Bound antibody was detected by incubation with a horseradish peroxidase (HRP)-goat anti-mouse IgG and color was developed with ABTS substrate (Pierce, Rockford, Ill.). OD was measured at 405 nm.

Dendritic cells. Human peripheral blood mononuclear cells (PBMC) from anonymous donors were obtained from separation cones of discarded apheresis units from the Coffee Memorial Blood Center, Amarillo, Tex. after platelet harvest. Cells were separated on a ficoll gradient (Amersham Biosciences, Uppsala, Sweden), then washed, counted, typed for HLA-A2 by flow cytometry, and resuspended in AIM-V medium at 1-2×10⁷ cells/ml. PBMC were incubated in a T-80 (Nalge-Nunc, Rochester, N.Y.) or T-175 (Corning, Acton, Mass.) flask, depending on the volume, for 2 h at 37° C. and 5% CO₂. Non-adherent cells were removed, the flask was washed twice with PBS, and then 15-30 ml supplemented AIM-V (10% heat-inactivated FBS, L-glutamine and Pen/Strep) was added to the flask, as well as IL-4 (50 ng/ml) and GM-CSF (25 ng/ml), stimulating differentiation of monocytes into dendritic cells. Recombinant human IL-4 and GM-CSF were obtained from Peprotech (Rockyhill, N.J.). After 5-6 days, the immature dendritic cells were detached from the flask by incubation at 4° C. for 20-60 min, centrifuged, counted and either used immediately or frozen at −80° C. for later use.

Peptide specificity and sensitivity assays. T2 is a mutant cell line that lacks transporter-associated proteins (TAP) 1 and 2 which allows for efficient loading of exogenous peptides (Wei et al., 1992). The T2 cells were pulsed with the peptides at 20 μg/ml for 4 h in growth medium with the exception of the peptide-titration experiments in which the peptide concentration was varied as indicated. Cells were washed and resuspended in staining buffer (SB; PBS+0.5% BSA+2 mM EDTA) and then stained with either a constant amount (1 μg) or a decreasing amount (4-0.1 μg) of 3F9 or 1B10 TCRm antibody for 15-30 min in 100 μl SB. Cells were then washed with 3 ml SB and the pellet was resuspended in 100 μl of SB containing 2 μl of either of two goat anti-mouse secondary antibodies (FITC or PE labeled). After incubating for 15-30 min at room temperature, the wash was repeated and cells were resuspended in 0.5 ml SB, analyzed on a FACScan instrument and evaluated using CELL QUEST™ Software (BD Biosciences, Franklin Lakes, N.J.). To evaluate the peptide binding sensitivity of each TCRm, T2 cells were pulsed for 4 h with decreasing amounts of specific peptide (2000-0.15 nM). T2 cells (5×10⁵) were then washed in SB to remove excess peptide and stained with each TCRm-PE conjugate, 3F9 and 1B10 TCRms at 1 μg/ml of SB).

Antigen presentation by vaccine-treated DCs using TcRm. Immature Dendritic cells were harvested and plated into 4 wells of a 24-well tissue culture plate. Either the vaccine (B11-hCGβ or the monoclonal antibody alone (“vehicle, B11”) were added at 30 μg/ml, two wells were untreated, and the plate was incubated for up to 3 days at 37° C., 5% CO₂. Cells were matured by addition of Poly I:C (Sigma, St. Louis, Mo.) at 50 ng/ml to the vaccine- and vehicle-treated well, as well as one of the untreated wells, then incubated for 12-18 h. Mature or immature (untreated) DCs were harvested as before, then centrifuged and divided into the appropriate number of aliquots for staining and analysis by flow cytometry.

Analysis of Ag-specific T cells by IFNγ and granzyme-B ELISpot assay. T cells were stimulated as bulk cultures in vitro on a 8-10 day cycle for 3-4 weeks with autologous immature DCs previously exposed to the vaccine (B11-hCGβ) and matured with Poly I:C) at a ratio of 10:1 in the presence of cytokines sequentially added (10 ng/ml each of IL-7 on day 0 and IL-2 on day 1) every 3 days. Alternatively, CD8+ T cells from HLA-A2+ donors were repeatedly stimulated with hCGβ synthetic peptides (TMTRVLQGV (SEQ ID NO:2) and GVLPALPQV (SEQ ID NO:4)) loaded on to matured autologous DCs. Effector T lymphocytes were expanded on anti-CD3 and anti-CD28 Dynal immunomagnetic beads (Invitrogen, Carlsbad, Calif.) and restimulated with vaccine on day 14 and CD8+ and CD4+ T cells were purified using a commercial T cell enrichment kit (Miltenyi MACS, Auburn, Calif.). CTL activity of vaccine or peptide-stimulated CD8+ T cells was assessed using vaccine treated DCs or peptide-loaded T2 cells in the presence of 3 μg/ml β2 microglobulin. CD8+CTL response was measured in a cell-based cytokine or granzyme-B production ELISpot assay (MabTech, Sweden and Cell Sciences, Canton, Mass. for ELISpot kits). Spot formation was evaluated by Dr. Sylvia Janetzki (Zellner Consulting, Inc., Fort Lee, N.J.). For inhibition experiments using TCRm, vaccine or vehicle-treated DCs were added to B11-hCGβ-specific CTL at a 1:1 ratio unless otherwise noted (see FIGS. 11 and 12). The TCRm mAbs were added (10 μg) to both vaccine- and vehicle-treated DCs+CTLs, and a mouse IgG1 isotype was also added as a control. Supernatant (100 μl/well) was collected at 24 and 48 h of incubation. Supernatant samples were frozen at −20° C. until testing was performed for Interferon-γ production using an IFNγ cytokine secretion assay (OptEIA™ Human IFN-γ ELISA Kit II, BD San Diego, Calif.).

Generation of HLA-class I peptide complexes. Soluble HLA-A*0101 and HLA-A*0201 complexes were prepared from inclusion bodies essentially as described by Altman et al. (1996). The human HLA-A*0101 and HLAA*0201 heavy chain genes, a kind gift from Dr. William Hildebrand (University of Oklahoma), were amplified by PCR and cloned into the pAC4 plasmid containing the birA amino acid sequence (Avidity, Denver, Colo.). The human beta-2 microglobulin gene was previously cloned into an expression vector for production in an E. coli strain BL-21 (Parker et al., 1989). Refolded monomer was concentrated and purified on an S-75 size exclusion column by FPLC (Pharmacia, Kalamazoo, Mich.) and then biotinylated using the biotin ligase enzyme according to the manufacturer's instructions (Avidity). Tetramers were formed by mixing the biotin tagged refolded HLA-A2-peptide complex with streptavidin at a molar ratio of 4-1, respectively. Tetramers were purified on an S-200 Sephadex size exclusion column and the protein concentration was determined by BCA protein assay (Pierce, Ill.). Soluble intact monomer of HLA-B*0702 protein was produced by LCL-721 B cell transfectants, purified by Protein-G and loaded with reticulocalbin-2 peptide₍₄ₐₐ₋₁₃ₐₐ₎ for use in ELISA.

FP-based peptide binding assay (PolyTest). Peptide binding experiments were performed on an Analyst™ AD Assay Detection System (Molecular Devices; Sunnyvale, Calif.) (Buchli et al., 2005; and Buchli et al., 2006). Briefly, each individual well of a black 96-well UL HE PS microplate (Molecular Devices) was loaded with 5 μl of an 8×β2m solution (160 μg/ml) (Fitzgerald Industries International; Concord, Mass.), 10 μl of 4× competitor at various dilutions, 5 μl of an 8×pFITC preparation (16 nM) and 20 μl of 2× activated sHLA (80 μg/ml). Soluble HLA was activated by incubating at 53° C. for 15 min. For all preparations, 1×BGG/PBS was used as buffer. Specific control groups included: (a) protein only, (b) tracer only, and (c) buffer only. The plate was incubated at room temperature and read periodically until no further increase in polarization was observed indicating that equilibrium was reached (24-48 h). FP values given as milli-polarization (mP) are calculated by the equation:polarization (mP)=1000(S−GP)/(S+GP), where S and P are background-subtracted intensities of the fluorescence measured in the parallel (S) and perpendicular (P) directions, respectively, and G (grating) is the instrument and assay dependent correction factor.

Competition experiments were analyzed by plotting FPₘₐₓ (maximal polarization) values as a function of the logarithms of competitor concentrations. The binding affinity of each competitor peptide was expressed as the concentration that inhibits 50% binding of the FITC-labeled reference peptide. Observed inhibitory concentrations (IC₅₀) were determined by nonlinear curve fitting to a dose-response model with variable slope using the specific software Prism (Graph Pad Software Inc.; San Diego, Calif.).

Statistical analysis. The relationship between two parameters was tested using regression analysis, and a value of p<0.05 was considered significant. In the presence of a significant relationship, the coefficient of determination (R²) was calculated to express the degree of correlation.

### Example 2

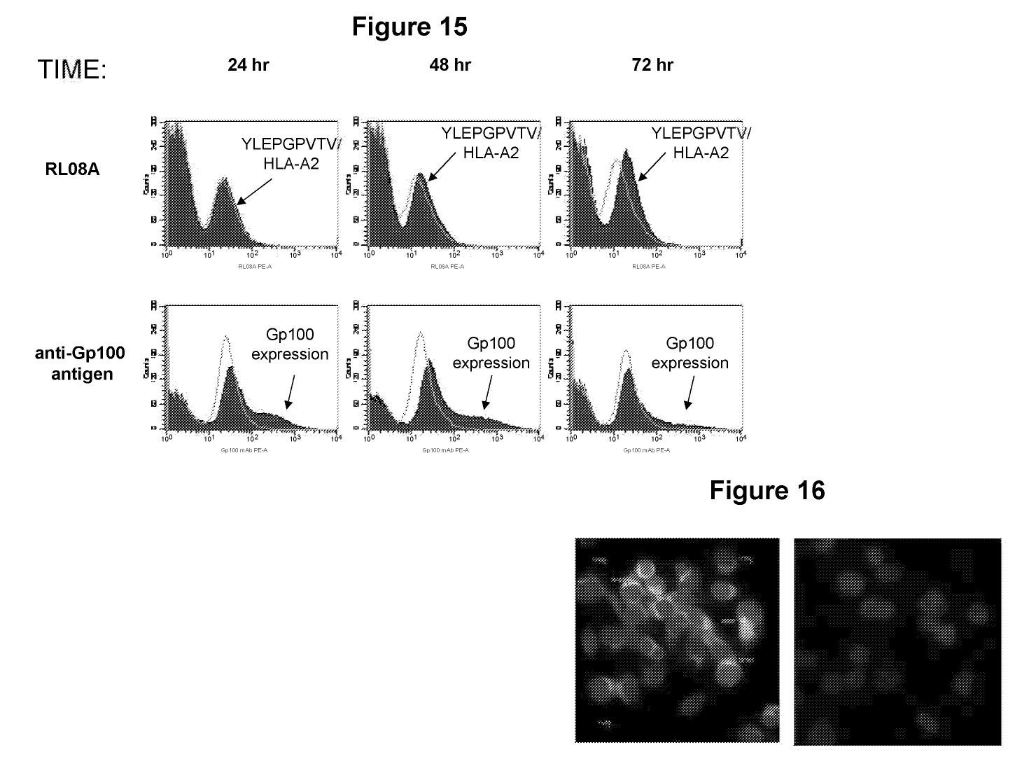

TCRm antibodies can readily detect de novo antigen processing and presentation in cells actively treated with an active immunotherapeutic (e.g., a vaccine composition) or from natural antigen expression (e.g., in virally infected or oncogenic tissues). These events can be tracked using flow cytometry staining as well as immunocytochemistry, with associated quantitation of observed values (FIGS. 15 and 16). An example of these studies is presented in FIG. 15, with data from control vaccine or target vaccine (Gp100 antigen) treated antigen presenting cells. There is a strong correlation between TCRm binding of HLA-peptide complexes present on the surface of vaccine treated cells and the presence of intracellular antigen. The temporal relationship between intracellular antigen detection and the appearance of specific HLA-peptide complexes will vary depending on the type of vaccine employed, e.g., peptide, intact protein, nucleic acid, viral vector, etc. Nevertheless, TCRm antibody binding activity correlates with intracellular antigen presence regardless of vaccine-type and properties.

The presentation of specific peptide epitopes on HLA molecules can be visualized by immunocytochemistry. In FIG. 16, cells processing Gp100 antigen are stained with the RL08A-TCRm or a control TCRm. Strong FITC fluorescence is observed on the surface of the cells in left panel where the TCRm has bound the appropriate peptide-HLA complex. The intensity of the fluorescence can be quantified allowing a measure of the number of HLA-peptide complexes to be determined (data not shown).

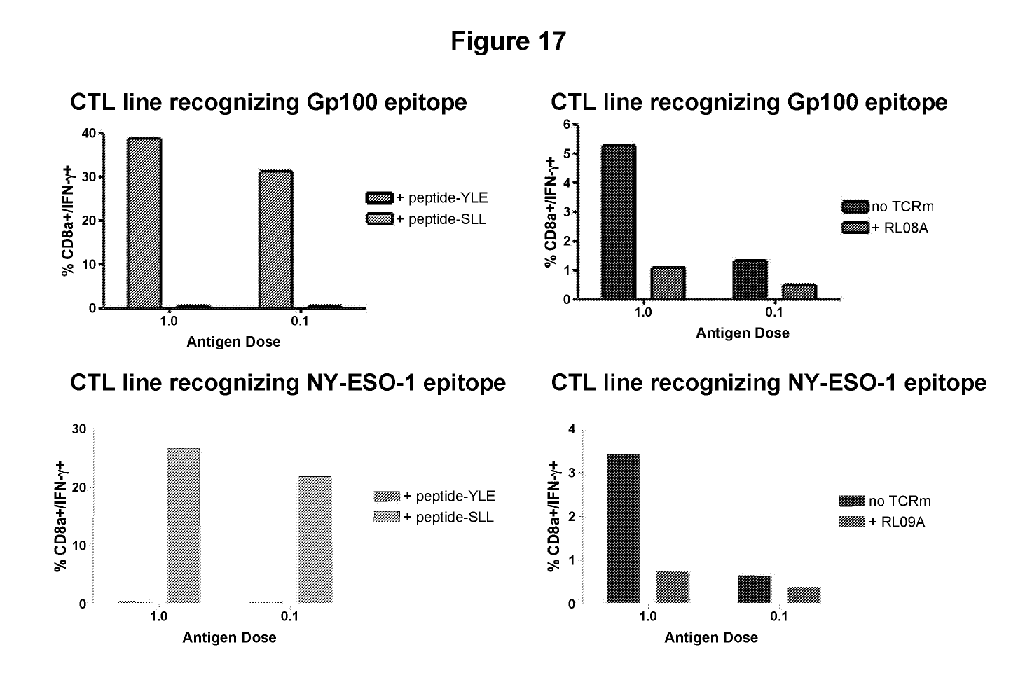

The sensitivity of TCRm binding of peptide-HLA complexes was compared with detection sensitivity observed in standard CTL-assays (using IFNg production as a surrogate; FIG. 17). Two separate batches of antigen presenting cells were incubated with Gp100 and NY-ESO-1 antigens respectively. The cells were then incubated with CTL lines recognizing either specific Gp100 or NY-ESO-1 antigen-peptide. Likewise, the cells were stained with RL08A-TCRm (recognizing Gp100 peptide-YLEPGPVTV; SEQ ID NO:75) and RL09A-TCRm (recognizing NY-ESO-1 peptide-SLLMWITQV; SEQ ID NO:13).

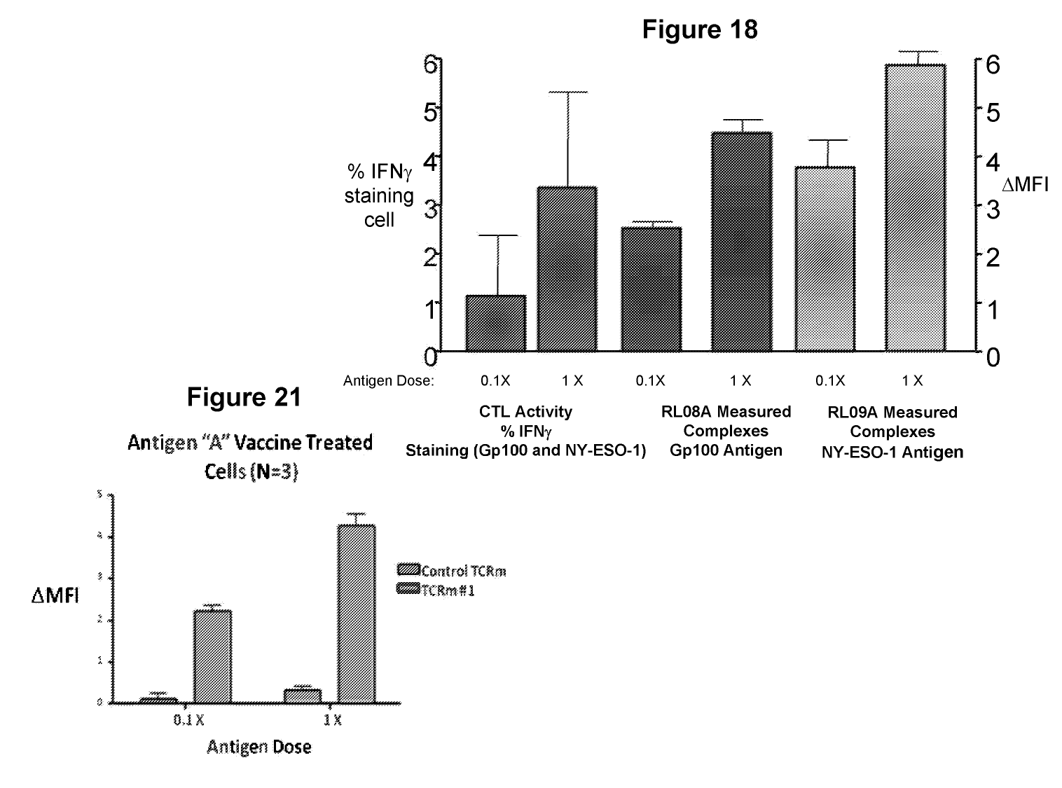

The data in FIG. 18 demonstrate that CTL stimulation and TCRm mAb binding intensity is antigen dose dependent and that both TCRm mAb's display detection sensitivity equivalent or better than the lower level sensitivity threshold for CTL lines. The conclusion drawn from these findings is that change in Mean Fluorescence Units as measured by TCRm staining is a sensitive and reproducible readout that correlates with CTL activity in vitro.

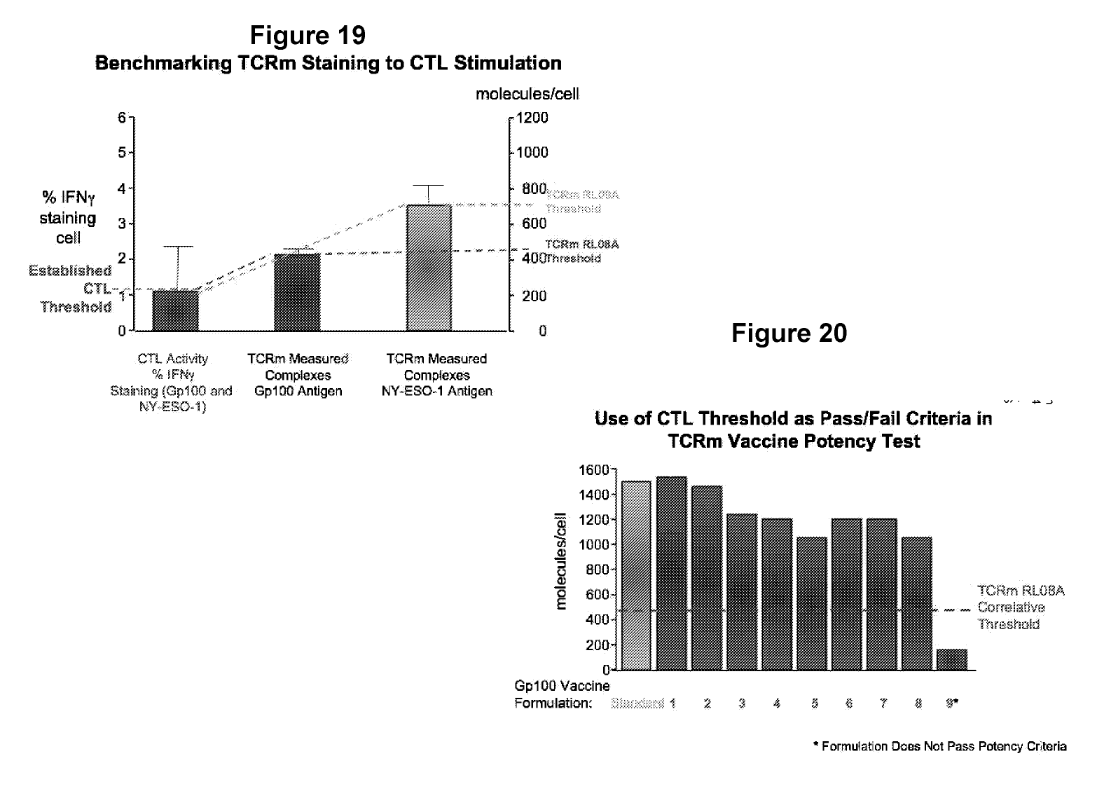

The induced CTL activities measured by incubating vaccine treated cells with appropriate CTL lines can be effectively correlated with quantitative measurement of peptide-MHC complexes on the surface of the vaccine treated cells as determined by TCRm antibodies. Data presented in FIG. 19 displays both CTL activity and TCRm staining data, thus allowing benchmarking of TCRm staining to CTL stimulation. Using vaccine dosing studies, the minimal acceptable CTL stimulation activity was determined (blue bar) and set as acceptance threshold value (blue dashed line) for both Vaccine Antigens gp100 and NYESO1. Parallel studies were carried out quantitating the number of specific HLA-peptid from gp100 and NYESO-1 complexes present on antigen presenting cells (purple and green bars, respectively). The complex numbers determined by TCRm staining of each antigen was determined at the threshold does of each vaccine. The Established CTL threshold was used to derive Correlative TCRm staining thresholds. The complex numbers measured by TCRm RL08A binding gp100-derived peptides for Vaccine containing gp100 Antigen at this Correlative threshold was ~450 HLA A*02-peptide a complexes (purple dashed line); by RL09A, for vaccine containing the NYESO-1 Antigen, this value was ~700 HLA A*02-peptide b complexes (green dashed line). These Correlative threshold values of complexes, benchmarked to CTL stimulation, now can be used to measure the potency of vaccine lots and formulations using appropriate archived standards.

These correlations can be effectively used to provide a pass/fail criteria for vaccine lots, formulations or instability testing assays, as shown in FIG. 20. The potency of nine different Gp100 Vaccine formulations were compared using the TCRm quantitative potency assays measuring the numbers of HLA-Gp100 peptide complexes. A Gp100 vaccine standard was used (left side in green) to compare the various vaccine formulations, and the CTL threshold for the Gp100 TCRm-RL08A, determined previously (FIG. 20), was used as the pass/fail benchmark for the formulations. Using this basis, formulations 1 through 8 were deemed acceptable, while formulation 9 failed based on the CTL activity threshold benchmark.

FIG. 21 shows data from three separate experiments using TCRm staining of gp100 vaccine treated cells, conducted with different antigen presenting cell populations during different weeks of study. The three studies show very small standard deviations, establishing the reproducibility of the TCRm binding assays. When one compares these standard deviations with those associated with the CTL assays presented in FIG. 18, one clearly sees the increased reproducibility and reliability of the data provided.

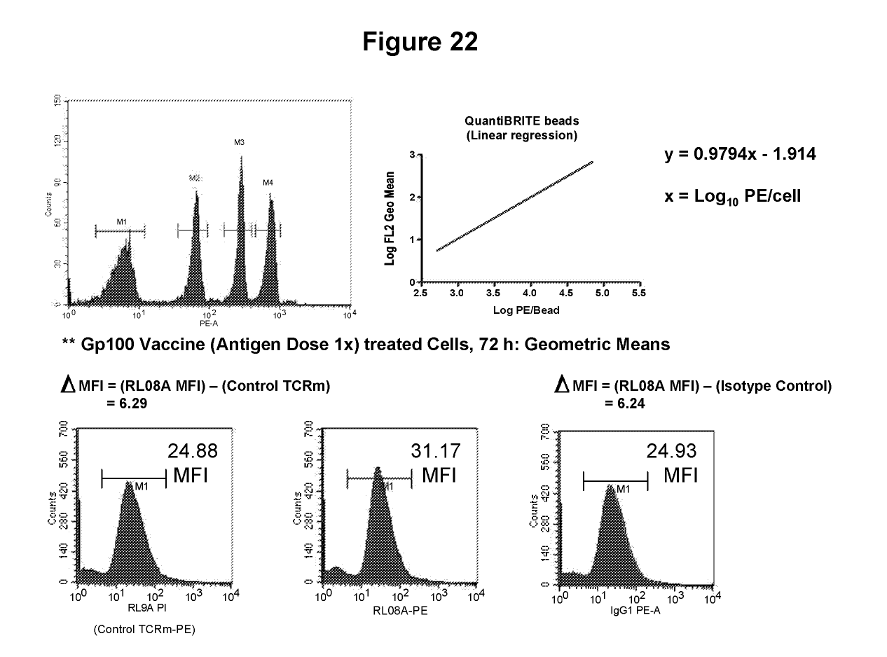

Using the QuantiBRITE™ system (BD Biosciences, Inc.), peak volumes from the flow cytometry plots and ΔMFI values can be used to determine the number of HLA-peptide complexes present in a given number of cells (FIG. 22). Standard materials with known quantities of PE molecules are supplied by manufacturer and separated using flow cytometry. The delta MFI values for these know samples are plotted and a linear regression formula is derived allowing unknown samples to be analyzed. The unknowns are reacted with a TCRm antibody and a secondary PE-labeled antibody which binds the TCRm antibody. This interaction will show a measureable delta MFI value. This value is placed in the regression formula, and a number of PE molecules correlating with this value is determined. Due to loading efficiency of our secondary antibodies at known amount of PE molecule(s) per antibody, this allows this number to establish the number of peptide-MHC complexes identified by the TCRm antibody.

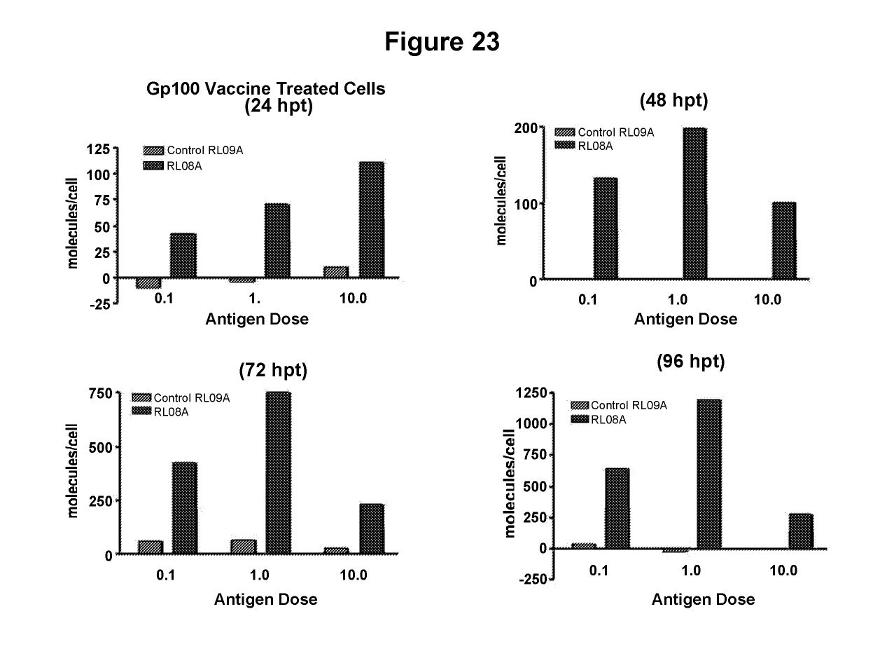

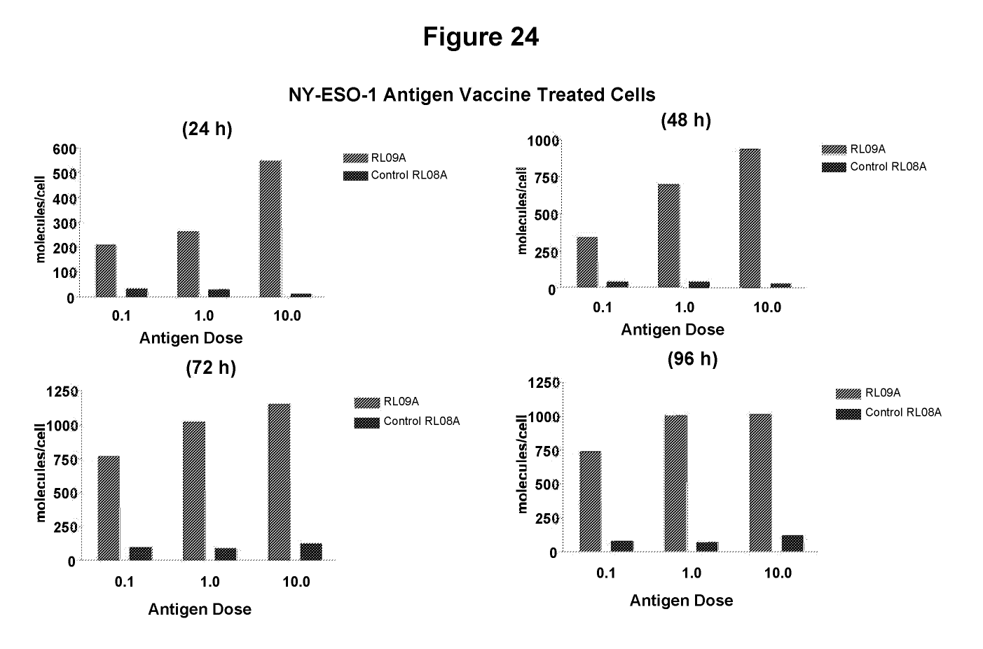

With knowledge of cell number, the average number of complexes per cell can be determined. FIGS. 23 and 24 show experiments investigating the temporal kinetics of HLA-peptide presentation. In separate experiments using three different vaccine doses, antigen presenting cells were treated with Gp100 and NY-ESO-1 antigens respectively and samples were taken at 24 h, 48 h, 72 h and 96 hours. Cells were incubated with both RL08A-TCRm and RL09A-TCRm and subjected to quantitative analysis as described above.

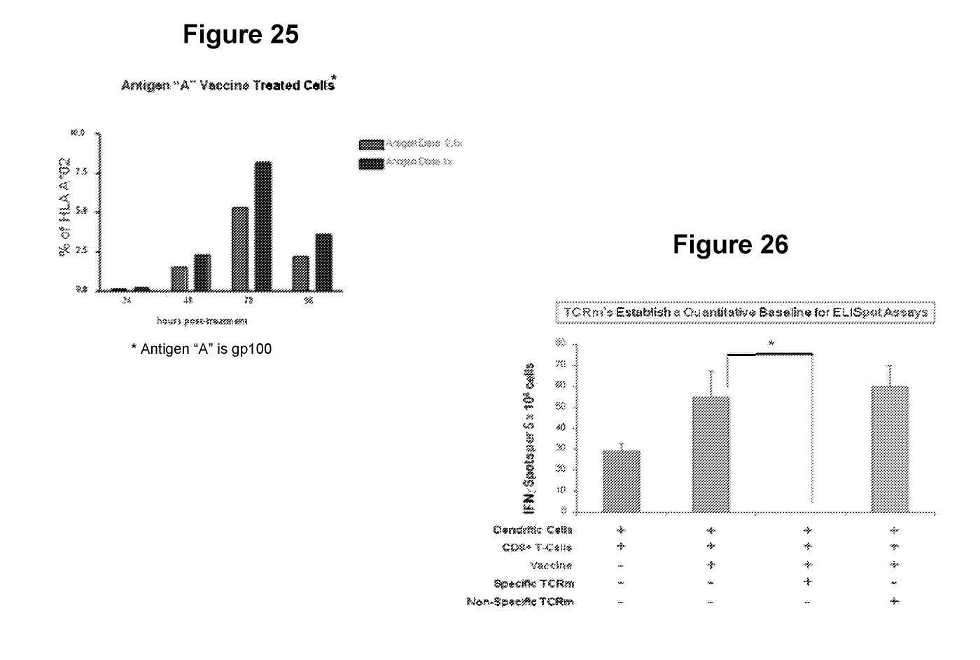

Further, simultaneous measurement of all HLA molecules on a given cell population with an HLA-specific antibody, such as BB7.1 which binds HLA A*02, and TCRm measurement of a specific peptide-HLA complex allows the percentage of HLA molecules occupied by a given antigen-specific peptide to be determined as shown in FIG. 25 using gp100 vaccine to treat cells.

The data presented demonstrate that TCRm antibody-based assays can be the basis for a quantitative, bio-potency assay for active immunotherapeutic products. Assays can be performed solely using TCRm antibodies. These assays are first benchmarked using CTL-specific activities and then performed in the absence of CTLs to provide reproducible, quantitative data concerning the potency of a given therapy preparation. These assay show the dynamic range required to quantitatively assess differences in therapeutic preparations. Potency differences can be compared with threshold values answering necessary quality questions. Further, TCRm antibodies assist with cell-based assays to remove assay background allowing more significant and comparable data to emerge. TCRm antibodies provide a highly sensitive and selective reagent, in a soluble and stable form, to empower accurate and quantitative measurement of potency of active immunotherapy drugs.

It has thus been demonstrated herein that the TCRm monoclonal antibody is an ideal biological tool for developing a quantitative bio-potency assay for CTL vaccines. The quantitative methodology using TCRm antibody staining has been developed, and a quantitative dynamic range has been demonstrated for peptide/HLA-A2 epitopes at <50 specific complexes on treated cells. Additionally, a quantitative dynamic range has been demonstrated for peptide/HLA-A2 epitopes at <2% of total HLA on treated cells. Further, CTL activities have been quantitatively correlated with TCRm's to same vaccine modality and dose. Therefore, a prototype quantitative bio-potency assay has been successfully established.

As a further example, in traditional ELISPOT assays, the background often observed in assays can be virtually eliminated by a pre-treatment with a TCRm antibody and completion of the normal assay. FIG. 26 shows the dramatic difference in assay significance with and without use of the TCRm antibody.

These data demonstrate the ability of TCRm antibodies to enhance the quality of established cell-based assays. Background in ELISpot, intracellular cytokine staining, and direct CTL assays (due to differences in operator, assay conditions and cell source) renders these assays semi-quantitative at best. Inclusion of TCRm antibodies in these cell-based assays can reduce this natural background and enhance the significance of individual assays, allowing assay comparability when performed at different times or with different samples. This is illustrated in FIG. 26, where the background present in an assay when dendritic cells are NOT treated with a vaccine is incubated with CD8+ T cells. This background makes the significance of the value seen with vaccine treated cells less impressive. Co-incubation of the vaccine treated cells with a TCRm specific to the complex produced blocks the background activities induced by the Tcells, reducing this level to virtually zero. Incubation of a TCRm antibody not specific to the induced vaccine complex results in no reduction in T-cell activities. This approach provides for a rapid manner to reduce background in assays and increase significance of the resulting data.

The Examples presented herein demonstrate that TCRm antibody-based assays can be the basis for a quantitative, biopotency assay for active immunotherapeutic products, eliminating the need for animal-based experimentation. Assays can be performed solely using TCRm antibodies. These assays are first benchmarked using CTL-specific activities and then performed in the absence of CTLs to provide reproducible, quantitative data concerning the potency of a given therapy preparation. These assays demonstrate the high reproducibility, dynamic range and specificity required to quantitatively assess differences in therapeutic preparations. Potency differences can be compared with threshold values answering necessary quality questions. Further, TCRm antibodies assist with cell-based assays to remove assay background allowing more significant and comparable data to emerge. TCRm antibodies provide a highly sensitive and selective reagent, in a soluble and stable form, to empower accurate and quantitative measurement of potency of active immunotherapy drugs.

Materials and Methods for Example 2

Cell line, culture technique, and viral vectors. The normal human male lung fibroblast cell line MRC-5 (ATCC CCL-171™) was cultured in BioWhittaker® EMEM (Lonza) supplemented with 2 mM HyQ® I-glutamine (HyClone), HyQ® penicillin-streptomycin solution (HyClone), and 10% Gibco™ Fetal Bovine Serum (FBS, Invitrogen Corp.). Cells were maintained in T-175 flasks and upon reaching confluence (approximately 8×10⁶ cells/flask) were trypsinized, washed, and subcultured at a 1:4 dilution. The ALVAC(2)-TRICOM viral vectors employed in MRC-5 infections consisted of vCP2264 (gp100/Mage1-3mini-hLFA-3/hICAM-1/hB7.1-vvE3L/vvK3L), vCP2292 (NY-ESO-1-hLFA-3/hICAM-1/hB7.1-vvE3L/vvK3L), and vCP2041 (hLFA-3/hICAM-1/hB7.1-vvE3L/vvK3L) provided by sanofi pasteur.

Peripheral blood mononuclear cell (PBMC) preparation. PBMCs were prepared via centrifugation of whole human blood diluted 1:1 in BioWhittaker® X-VIVO-10™ (Lonza) medium over Ficoll-Paque™ PLUS (GE Healthcare). Separations were carried out in 50 mL conical tubes containing 35 mL of the blood dilution and 15 mL of Ficoll-Paque™ PLUS. Cells collected from the interface were counted, washed twice, and then frozen down in 1.5 mL aliquots of 5×10⁷ cells in 90% FBS with 10% DMSO (Fisher Scientific) and stored at −80° C. until use.

Dendritic cell (DC) generation. Non-manipulated monocytes were purified from PBMCs using the human Monocyte Isolation Kit II (Miltenyi Biotec Inc.) according to the manufacturer's instructions. DCs were then generated as previously described (1). Briefly, monocytes were cultured in 24-well plates at 5×10⁵ cells/well in 1 mL volumes of BioWhittaker® RPMI (Lonza) supplemented with I-glutamine, penicillin-streptomycin solution, 10% human AB (hAB) serum (Valley Biomedical, Inc.), 100 ng/mL recombinant human GM-CSF (R&D Systems), and 200 ng/mL recombinant human IL-15 (R&D Systems). Immature DC were activated on day 3 by the addition of LPS (E. coli strain 026:B6, Sigma) at a concentration of 10 ng/mL and used as mature DCs on day 4.

Cytotoxic T lymphocyte (CTL) line priming with peptide-pulsed DCs. Non-manipulated CD8⁺ T cells were purified from autologous PBMCs using the human CD8⁺ T Cell Isolation Kit II (Miltenyi Biotec Inc.) according to the manufacturer's instructions. Mature DCs were treated with 10 μg/mL mitomycin C (Sigma) for 45 min at 37° C., washed twice, and loaded in the presence of 3 μg/mL purified human beta-2-microglobulin (β₂m, Lee Biosolutions, Inc.) with 10 μg/mL of either YLEPGPVTV peptide (gp100-derived epitope; SEQ ID NO:75) or SLLMWITQV peptide (NY-ESO-1-derived epitope; SEQ ID NO:13) for 2 h in the 24-well plates at 37° C. CD8⁺ T cells were then added at 1×10⁶ cells/well in 1 mL volumes of RPMI/10% hAB containing 10 IU/mL recombinant human IL-7 (R&D Systems) and placed at 37° C. for 7 days.

CTL line restimulation with peptide-pulsed adherent antigen-presenting cells (APCs). Adherent APCS were prepared from autologous PBMCs and used to restimulate CTL lines essentially as described (2). In brief, 4×10⁶ mitomycin C-treated PBMCs were added per well to 24-well plates in 0.5 mL volumes of RPMI/10% hAB and incubated for 2 to 3 hours at 37° C. for adherence. The media was then carefully removed and replaced with 0.5 mL fresh media containing 3 μg/mL β₂m and 10 μg/mL of the relevant peptide for 2 h at 37° C. After washing once with media to remove excess peptide, CTLs harvested from either initial priming or previous restimulation were added at 1×10⁶ cells/well in 1 mL volumes of RPMI/10% hAB containing 10 IU/mL recombinant human IL-2 (R&D Systems). The cultures were fed every 3-4 days with 0.5 mL fresh media containing IL-2 and restimulated at 7-10 days.

Viral infection of MRC-5. MRC-5 cells were seeded in 6-well plates at 2×10⁵ cells/well (2 mL/well) approximately 24 h prior to infection. An extra plate was seeded for the purposes of harvesting and counting prior to infection for MOI calculations. Virus stocks were thawed at room temperature from −80° C. storage and then kept on ice. Aliquots (30 μL) were sonicated on ice water using a Misonix S-4000 sonicator (amplitude: 20, process time: 5 s, pulse-on: 1 s, pulse-off: 3s), diluted 1:100 in MRC-5 medium, and then further diluted to provide the desired MOI in a deliverable volume of 1 mL/well. The MRC-5 plates were infected by removing all media from the wells and adding 1 mL/well of diluted virus. The plates were placed at 37° C./5% CO₂ for 2 h, during which time they were gently shaken every 15 min. The infection was stopped by adding 2 mL/well of MRC-5 medium, and the plates were returned to the incubator for 72 h.

Intracellular staining of MRC-5 with gp100 and NY-ESO-1 monoclonal antibodies (MAbs). MRC-5 cells were harvested from 6-well plates by removing all media, adding 1 mL/well Cellgro® Trysin EDTA (Mediatech Inc.), and incubating at 37° C. for 2-3 minutes; 2 mL/well of RPMI/10% hAB was then added per well and the cells collected. The cells were washed, resuspended in 5 mL of RPMI/10% hAB, and counted; they were maintained in human serum-containing medium for at least 10 min prior to staining in order to block non-specific binding sites. Once the assay layout for staining in 96-well U-bottom plates was established, between 3×10⁵ to 5×10⁵ cells/well were plated, spun down, and resuspended in 100 μL/well of BD Cytofix/Cytoperm™ Fixation/Permeabilization solution (BD Biosciences) and incubated at room temperature (RT) for 20 min. Next, 100 μL/well of BD Perm/Wash™ buffer (BD Biosciences) was added and the plates centrifuged. The cells were then washed twice with 200 μL/well of Perm/Wash™ buffer. Primary antibodies (anti-tumour antigen MAbs) were added to indicated wells in 100 μL volumes of Perm/Wash™ buffer at the following concentrations: HMB45 (anti-gp100, Signet Laboratories, Inc.), 1:100; E978 (anti-NY-ESO-1, Santa Cruz Biotechnology), 500 ng/mL. The plates were incubated at RT for 40 min, after which they were washed as before (100 μL Perm/Wash™ buffer added per well and spun, followed by two washes with 200 μL/well Perm/Wash™ buffer). Secondary antibody was added to indicated wells in 100 μL volumes of PermWash buffer at 1 μL PE-labeled rat anti-mouse IgG1 (A85-1, BD Bioscience) per well. The plates were incubated in the dark at RT for 30 min, after which they were washed as before. The plates were then washed twice with 200 μL/well of FACS buffer (PBS containing 5% FBS and 2 mM EDTA) prior to transferring samples into tubes for data acquisition on a BD FACSCanto (BD Biosciences).

Surface staining of MRC-5 with TCRms. MRC-5 cells were harvested, counted, and incubated in RPMI/10% hAB as described above. Once the assay layout for staining in 96-well U-bottom plates was established, between 3×10⁵ to 5×10⁵ cells/well were plated, spun down, and resuspended in 100 μL/well of FACS buffer. Primary antibodies (TCRms) were added to indicate wells in 100 μL volumes of FACS buffer at final concentrations of 250 ng/mL. The plates were incubated on ice for 30 min, after which they were spun and then washed twice with 200 μL/well FACS buffer. Secondary antibody was added to indicated wells in 200 μL volumes of FACS buffer at 1 μL PE-labeled rat anti-mouse IgG1 per well. The plates were incubated in the dark on ice for 20 min, after which they were washed as before. Samples were then transferred into tubes for data acquisition on a FACSCanto.

Intracellular staining of CTL stimulated by virally-infected MRC-5 with an IFN- [symbol] MAb. CTL lines were harvested, washed, and resuspended in MRC-5 medium containing 1 μL/mL of BD GolgiPlug (BD Bioscience) at day 7 before adding 4×10⁶ cells/well in 3 mL volumes to 72 h cultures of infected MRC-5 cells in 6-well plates. For relevant and irrelevant peptide-pulsed controls, MRC-5 cells were pulsed with 10 μg/mL of peptide for 2 h at 37° C. prior to addition of CTLs. TCRm blockade was accomplished through pre-incubation of MRC-5 cells with 10 μg/mL of the corresponding TCRm for 30 min at 37° C. CTLs were incubated with MRC-5 for 5 h at 37° C. and then harvested. Once the assay layout for staining in 96-well U-bottom plates was established, between 7×10⁵ to 8×10⁵ cells/well were plated, spun down, and resuspended in 100 μL/well of FACS buffer containing 20 μL/well of APC-labeled anti-human CD8a (RPA-T8, eBioscience). The plates were incubated in the dark on ice for 20 min, after which 100 μL/well of FACS buffer was added and the plates centrifuged. The cells were then washed once with 200 μL/well of FACS buffer prior to resuspension in 100 μL/well of BD Cytofix/Cytoperm™ Fixation/Permeabilization solution and incubation on ice for 20 min. The cells were then washed in BD Perm/Wash™ buffer as described above for intracellular staining of MRC-5. The PE-labeled anti-human IFN-γ antibody (4S.B3, eBioscience) was added to indicated wells in 100 μL volumes of Perm/Wash buffer at a concentration of 1 μL per well. The plates were incubated in the dark on ice for 30 min, after which they were washed as before. After washing twice in 200 μL/well of FACS buffer, the samples were transferred into tubes for data acquisition on a FACSCanto.

Thus, in accordance with the presently disclosed and claimed inventive concept(s), there has been provided a method of assaying potency of a vaccine composition that fully satisfies the objectives and advantages set forth hereinabove. Although the inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the inventive concept(s).

### REFERENCES

The following references, to the extent that they provide exemplary procedural or other details supplementary to those set forth herein, are specifically incorporated herein by reference.
Adotevi O, Mollier K, Neuveut C, Cardinaud S, Boulanger E, Mignen B, et al. Immunogenic HLA-B*0702-restricted epitopes derived from human telomerase reverse transcriptase that elicit antitumor cytotoxic T-cell responses. Clin Cancer Res 2006; 12(10):3158-67.
Altman J D, Moss P A, Goulder P J, Barouch D H, McHeyzer-Williams M G, Bell J I, et al. Phenotypic analysis of antigen-specific T lymphocytes. Science 1996; 274(5284):94-6.
Andersen P S, Stryhn A, Hansen B E, Fugger L, Engberg J, Buus S. A recombinant antibody with the antigen-specific, major histocompatibility complex-restricted specificity of T cells. Proc Natl Acad Sci USA 1996; 93(5):1820-4.
Bonifaz L, Bonnyay D, Mahnke K, Rivera M, Nussenzweig M C, Steinman R M. Efficient targeting of protein antigen to the dendritic cell receptor DEC-205 in the steady state leads to antigen presentation on major histocompatibility complex class I products and peripheral CD8+ T cell tolerance. J Exp Med 2002; 196(12):1627-38.
Buchli R, Vangundy R S, Giberson C F, Hildebrand W H. Critical factors in the development of fluorescence polarization-based peptide binding assays: an equilibrium study monitoring specific peptide binding to soluble HLA-A*0201. J Immunol Methods 2006; 314(1/2):38-53.
Buchli R, VanGundy R S, Hickman-Miller H D, Giberson C F, Bardet W, Hildebrand W H. Development and validation of a fluorescence polarization-based competitive peptide-binding assay for HLA-A*0201—a new tool for epitope discovery. Biochemistry 2005; 44(37):12491-507.
Buchli R, VanGundy R S, Hickman-Miller H D, Giberson C F, Bardet W, Hildebrand W H. Real-time measurement of in vitro peptide binding to soluble HLA-A*0201 by fluorescence polarization. Biochemistry 2004; 43(46):14852-63.
Bullock T N, Colella T A, Engelhard V H. The density of peptides displayed by dendritic cells affects immune responses to human tyrosinase and gp100 in HLA-A2 transgenic mice. J Immunol 2000; 164(5):2354-61.
Bullock T N, Mullins D W, Engelhard V H. Antigen density presented by dendritic cells in vivo differentially affects the number and avidity of primary, memory, and recall CD8+ T cells. J Immunol 2003; 170(4):1822-9.
Cells E., V. Tsai, C. Crimi, R. DeMars, P. A. Wentworth, R. W. Chesnut, H. M. Grey, A. Sette, and H. M. Serra. “Induction of anti-tumor cytotoxic T lymphocytes in normal humans using primary cultures and synthetic peptide epitopes”. Proc. Natl. Acad. Sci. USA 1994; 91:2105-2109.
Chiriva-Internati M, Liu Y, Weidanz J A, Grizzi F, You H, Zhou W, et al. Testing recombinant adeno-associated virus-gene loading of dendritic cells for generating potent cytotoxic T lymphocytes against a prototype self-antigen, multiple myeloma HM1.24. Blood 2003; 102(9):3100-7.
Clark C E, Vonderheide R H. Getting to the surface: a link between tumor antigen discovery and natural presentation of peptide-MHC complexes. Clin Cancer Res 2005; 11(15):5333-6.
Dangles V, Halberstam I, Scardino A, Choppin J, Wertheimer M, Richon S, et al. Tumor-associated antigen human chorionic gonadotropin beta contains numerous antigenic determinants recognized by in vitro-induced CD8+ and CD4+ T lymphocytes. Cancer Immunol Immunother 2002; 50(12):673-81.
Denkberg G, Cohen C J, Lev A, Chames P, Hoogenboom H R, Reiter Y. Direct visualization of distinct T cell epitopes derived from a melanoma tumor-associated antigen by using human recombinant antibodies with MHC-restricted T cell receptor like specificity. Proc Natl Acad Sci USA 2002; 99(14):9421-6.
Dubsky P., H. Saito, M. Leogier, C. Dantin, J. E. Connolly, J. Banchereau, and A. K. Palucka. “IL-15-induced human DC efficiently prime melanoma-specific naïve CD8+ T cells to differentiate into CTL”. Eur. J. Immunol. 2007; 37:1678-1690.
Gauduin M C. Intracellular cytokine staining for the characterization and quantitation of antigen-specific T lymphocyte responses. Methods 2006; 38(4):263-73.
Godelaine D, Carrasco J, Lucas S, Karanikas V, Schuler-Thurner B, Coulie P G, et al. Polyclonal CTL responses observed in melanoma patients vaccinated with dendritic cells pulsed with a MAGE-3.A1 peptide. J Immunol 2003; 171(9):4893-7.
He L Z, Ramakrishna V, Connolly J E, Wang X T, Smith P A, Jones C L, et al. A novel human cancer vaccine elicits cellular responses to the tumor-associated antigen, human chorionic gonadotropin beta. Clin Cancer Res 2004; 10(6):1920-7.
Hersey P, Menzies S W, Coventry B, Nguyen T, Farrelly M, Collins S, et al. Phase I/II study of immunotherapy with T-cell peptide epitopes in patients with stage IV melanoma. Cancer Immunol Immunother 2005; 54(3):208-18.
Hinz T, Buchholz C J, van der Stappen T, Cichutek K, Kalinke U. Manufacturing and quality control of cell-based tumor vaccines: a scientific and a regulatory perspective. J Immunother 2006; 29(5):472-6.
Itoh K, Yamada A. Personalized peptide vaccines: a new therapeutic modality for cancer. Cancer Sci 2006; 97(10):970-6.
Kageyama S, Tsomides T J, Sykulev Y, Eisen H N. Variations in the number of peptide-MHC class I complexes required to activate cytotoxic T cell responses. J Immunol 1995; 154(2):567-76.
Keilholz U, Martus P, Scheibenbogen C. Immune monitoring of T-cell responses in cancer vaccine development. Clin Cancer Res 2006; 12(7 Part 2):2346s-52s.
Keilholz U, Weber J, Finke J H, Gabrilovich D I, Kast W M, Disis M L, et al. Immunologic monitoring of cancer vaccine therapy: results of a workshop sponsored by the Society for Biological Therapy. J Immunother 2002; 25(2): 97-138.
Kukutsch N A, Rossner S, Austyn J M, Schuler G, Lutz M B. Formation and kinetics of MHC class I-ovalbumin peptide complexes on immature and mature murine dendritic cells. J Invest Dermatol 2000; 115(3):449-53.
Lev A, Denkberg G, Cohen C J, Tzukerman M, Skorecki K L, Chames P, et al. Isolation and characterization of human recombinant antibodies endowed with the antigen-specific, major histocompatibility complex-restricted specificity of T cells directed toward the widely expressed tumor T-cell epitopes of the telomerase catalytic subunit. Cancer Res 2002; 62(11):3184-94.
Lou Y, Wang G, Lizee G, Kim G J, Finkelstein S E, Feng C, et al. Dendritic cells strongly boost the antitumor activity of adoptively transferred T cells in vivo. Cancer Res 2004; 64(18):6783-90.
Markovic S N, Suman V J, Ingle J N, Kaur J S, Pitot H C, Loprinzi C L, et al. Peptide vaccination of patients with metastatic melanoma: improved clinical outcome in patients demonstrating effective immunization. Am J Clin Oncol 2006; 29(4):352-60.
Mosca P J, Hobeika A C, Clay T M, Morse M A, Lyerly H K. Direct detection of cellular immune responses to cancer vaccines. Surgery 2001; 129(3):248-54.
Nakamura M, Iwahashi M, Nakamori M, Ueda K, Ojima T, Naka T, et al. Dendritic cells transduced with tumor-associated antigen gene elicit potent therapeutic antitumor immunity: comparison with immunodominant peptide-pulsed DCs. Oncology 2005; 68(2/3):163-70.
Novellino L, Castelli C, Parmiani G. A listing of human tumor antigens recognized by T cells: March 2004 update. Cancer Immunol Immunother 2005; 54(3):187-207.
Oka Y, Tsuboi A, Kawakami M, Elisseeva O A, Nakajima H, Udaka K, et al. Development of W T1 peptide cancer vaccine against hematopoietic malignancies and solid cancers. Curr Med Chem 2006; 13(20):2345-52.
Parker K C, Wiley D C. Overexpression of native human beta 2-microglobulin in Escherichia coli and its purification. Gene 1989; 83(1):117-24.
Petricciani J, Egan W, Vicari G, Furesz J, Schild G. Potency assays for therapeutic live whole cell cancer vaccines. Biologicals 2006.
Porgador A, Yewdell J W, Deng Y, Bennink J R, Germain R N. Localization, quantitation, and in situ detection of specific peptide-MHC class I complexes using a monoclonal antibody. Immunity 1997; 6(6):715-26.
Ramakrishna V, Treml J F, Vitale L, Connolly J E, O'Neill T, Smith P A, et al. Mannose receptor targeting of tumor antigen pmel17 to human dendritic cells directs anti-melanoma T cell responses via multiple HLA molecules. J Immunol 2004; 172(5):2845-52.
Steinman R M. Dendritic cells and immune-based therapies. Exp Hematol 1996; 24(8):859-62.
Wei M L, Cresswell P. HLA-A2 molecules in an antigen-processing mutant cell contain signal sequence-derived peptides. Nature 1992; 356(6368):443-6.
Weidanz J A, Nguyen T, Woodburn T, Neethling F A, Chiriva-Internati M, Hildebrand W H, et al. Levels of specific peptide—HLA class I complex predicts tumor cell susceptibility to CTL killing. J Immunol 2006; 177(8):5088-97.
Weidanz J A, Piazza P, Hickman-Miller H, Woodburn T, Nguyen T, Wahl A, et al. Development and implementation of a direct detection, quantitation and validation system for class I MHC self-peptide epitopes. J Immunol Methods 2007; 318:47-58.
Wherry E J, McElha ugh M J, Eisenlohr L C. Generation of CD8 (+) T cell memory in response to low, high, and excessive levels of epitope. J Immunol 2002; 168(9):4455-61.
Wherry E J, Puorro K A, Porgador A, Eisenlohr L C. The induction of virus-specific CTL as a function of increasing epitope expression: responses rise steadily until excessively high levels of epitope are attained. J Immunol 1999; 163(7):3735-45.
Whiteside T L, Gooding W. Immune monitoring of human gene therapy trials: potential application to leukemia and lymphoma. Blood Cells Mol Dis 2003; 31(1):63-71.
Whiteside T L, Zhao Y, Tsukishiro T, Elder E M, Gooding W, Baar J. Enzyme-linked immunospot, cytokine flow cytometry, and tetramers in the detection of T-cell responses to a dendritic cell-based multipeptide vaccine in patients with melanoma. Clin Cancer Res 2003; 9(2):641-9.
Wittman V P, Woodburn D, Nguyen T, Neethling F A, Wright S, Weidanz J A. Antibody targeting to a class I MHC-peptide epitope promotes tumor cell death. J Immunol 2006; 177(6):4187-95.
Xia D, Moyana T, Xiang J. Combinational adenovirus-mediated gene therapy and dendritic cell vaccine in combating well established tumors. Cell Res 2006; 16(3):241-59.
Yang S, Linette G P, Longerich S, Haluska F G. Antimelanoma activity of CTL generated from peripheral blood mononuclear cells after stimulation with autologous dendritic cells pulsed with melanoma gp100 peptide G209-2M is correlated to TCR avidity. J Immunol 2002; 169(1):531-9.
Yang S, Linette G P, Longerich S, Roberts B L, Haluska F G. HLAA2.1/K (b) transgenic murine dendritic cells transduced with an adenovirus encoding human gp100 process the same A2.1-restricted peptide epitopes as human antigen-presenting cells and elicit A2. 1-restricted peptide-specific CTL. Cell Immunol 2000; 204(1):29-37.
Yee C, Savage P A, Lee P P, Davis M M, Greenberg P D. Isolation of high avidity melanoma-reactive CTL from heterogeneous populations using peptide-MHC tetramers. J Immunol 1999; 162(4):2227-34.
Zehn D, Cohen C J, Reiter Y, Walden P. Efficiency of peptide presentation by dendritic cells compared with other cell types: implications for cross-priming. Int Immunol 2006.
Zehn D, Cohen C J, Reiter Y, Walden P. Extended presentation of specific MHC-peptide complexes by mature dendritic cells compared to other types of antigen-presenting cells. Eur J Immunol 2004; 34(6):1551-60.

1-20. (canceled)

21. A method of assaying the potency of a vaccine composition, the method comprising the steps of:
delivering a vaccine composition to at least one antigen presenting cell so that the antigen presenting cell displays a peptide/MHC complex having an epitope derived from the vaccine composition;
contacting the at least one antigen presenting cell with a T cell receptor mimic (TCRm) antibody or fragment thereof reactive against the peptide/MHC complex;
quantitatively measuring the number of peptide/MHC complexes bound by the TCRm antibody or fragment thereof; and
determining the potency of the vaccine composition based on the number of peptide/MHC complexes bound by the TCRm antibody or fragment thereof.

22. The method of claim 21, wherein the at least one antigen presenting cell is a dendritic cell.

23. The method of claim 21, wherein the vaccine composition comprises a protein vaccine.

24. The method of claim 21, wherein the vaccine composition comprises a peptide vaccine.

25. The method of claim 21, wherein the TCRm antibody or fragment thereof is reactive to HLA-A2 MHC complexes.

26. The method of claim 21, wherein the step of quantitatively measuring the number of peptide/MHC complexes comprises measuring less than 60 complexes per cell.

27. The method of claim 21, wherein the step of determining the potency of the vaccine composition further comprises determining the level of antigen specific T cell stimulation.

28. The method of claim 27, wherein the step of determining the level of antigen specific T cell stimulation comprises determining the level of cytotoxic T cell (CTL) stimulation.

29. The method of claim 21, wherein the TCRm antibody or fragment thereof has a binding affinity for the specific peptide/MHC complex of about 10 nanomolar or greater.

30. The method of claim 21, wherein the step of quantitatively measuring the number of peptide/MHC complexes bound by the TCRm antibody or fragment thereof comprises detecting a label linked to the TCRm antibody or fragment thereof.

31. The method of claim 21, wherein the step of quantitatively measuring the number of peptide/MHC complexes bound by the TCRm antibody or fragment thereof comprises flow cytometry.

32. A method of determining the potency of a vaccine composition, the method comprising the steps of:
contacting antigen presenting cells with a vaccine composition;
contacting the antigen presenting cells with a T cell receptor mimic (TCRm) antibody or fragment thereof, wherein the antigen presenting cells display a peptide/MHC complex having an epitope derived from the vaccine composition and wherein the TCRm antibody or fragment thereof specifically binds the peptide/MHC complex;
measuring the number of peptide/MHC complexes bound by the T cell receptor mimic antibody; and
determining the potency of the vaccine composition based on the number of peptide/MHC complexes bound by the TCRm antibody or fragment thereof.

33. The method of claim 32, wherein the antigen presenting cells are dendritic cells.

34. The method of claim 32, wherein the vaccine composition comprises a protein vaccine.

35. The method of claim 32, wherein the vaccine composition comprises a peptide vaccine.

36. The method of claim 32, wherein the TCRm antibody or fragment thereof is reactive to HLA-A2 MHC complexes.

37. The method of claim 32, wherein the step of measuring the number of peptide/MHC complexes comprises measuring less than 60 complexes per cell.

38. The method of claim 32, wherein the step of determining the potency of the vaccine composition further comprises determining the level of antigen specific T cell stimulation.

39. The method of claim 38, wherein the step of determining the level of antigen specific T cell stimulation comprises determining the level of cytotoxic T cell (CTL) stimulation.

40. The method of claim 32, wherein the TCRm antibody or fragment thereof has a binding affinity for the specific peptide/MHC complex of about 10 nanomolar or greater.

41. The method of claim 32, wherein the step of measuring the number of peptide/MHC complexes bound by the TCRm antibody or fragment thereof comprises detecting a label linked to the TCRm antibody or fragment thereof.

42. The method of claim 32, wherein the step of measuring the number of peptide/MHC complexes bound by the TCRm antibody or fragment thereof comprises flow cytometry.
